SEQUENCE LISTING (130 sequences)

SEQ ID NO: 1 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 2351 aa, Artificial Sequence
  Description of Artificial Sequence: Synthetic polypeptide
  MOD_RES (608)..(626): Any amino acid
  MOD_RES (2213)..(2240): Any amino acid
  See specification as filed for detailed description of substitutions and preferred embodiments
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTXXXXXXXXXXXXXXXXXXXFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASXXXXXXXXXXXXXXXXXXXXXXXXXXXXQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 20 aa, Homo sapiens
SYFTNMFATWSPSKARLHLQ

SEQ ID NO: 4 - protein, 12 aa, Homo sapiens
SYFTNMFATWSP

SEQ ID NO: 5 - protein, 20 aa, Homo sapiens
TWSPSKARLHLQGRSNAWRP

SEQ ID NO: 6 - protein, 19 aa, Homo sapiens
ENIQRFLPNPAGVQLEDPE

SEQ ID NO: 7 - protein, 9 aa, Homo sapiens
FLPNPAGVQ

SEQ ID NO: 8 - DNA, 33 bp, Artificial Sequence
  Description of Artificial Sequence: Synthetic primer
ggcgcgcatatggatttaaatagttgcagcatg

SEQ ID NO: 9 - DNA, 30 bp, Artificial Sequence
  Description of Artificial Sequence: Synthetic primer
ggcgcgggatccctagtagaggtcctgtgc

SEQ ID NO: 10 - protein, 10 aa, Artificial Sequence
  Description of Artificial Sequence: Synthetic 10xHis tag
HHHHHHHHHH

SEQ ID NO: 11 - DNA, 45 bp, Artificial Sequence
  Description of Artificial Sequence: Synthetic primer
ggatttaaatagttgcgccatgccattgggaatggagagtaaagc

SEQ ID NO: 12 - DNA, 45 bp, Artificial Sequence
  Description of Artificial Sequence: Synthetic primer
ggatttaaatagttgcagcgcgccattgggaatggagagtaaagc

SEQ ID NO: 13 - DNA, 40 bp, Artificial Sequence
  Description of Artificial Sequence: Synthetic primer
gcatgccattgggaatggcgagtaaagcaatatcagatgc

SEQ ID NO: 14 - DNA, 43 bp, Artificial Sequence
  Description of Artificial Sequence: Synthetic primer
gcagcatgccattgggaatggaggctaaagcaatatcagatgc

SEQ ID NO: 15 - DNA, 42 bp, Artificial Sequence
  Description of Artificial Sequence: Synthetic primer
gggaatggagagtaaagcaatagcagatgcacagattactgc

SEQ ID NO: 16 - DNA, 42 bp, Artificial Sequence
  Description of Artificial Sequence: Synthetic primer
gggaatggagagtaaagcaatatcagctgcacagattactgc

SEQ ID NO: 17 - DNA, 42 bp, Artificial Sequence
  Description of Artificial Sequence: Synthetic primer
gcaatatcagatgcacagattgctgcttcatcctactttacc

SEQ ID NO: 18 - DNA, 45 bp, Artificial Sequence
  Description of Artificial Sequence: Synthetic primer
gcacagattactgcttcatccgcctttaccaatatgtttgccacc

SEQ ID NO: 19 - DNA, 45 bp, Artificial Sequence
  Description of Artificial Sequence: Synthetic primer
gcacagattactgcttcatcctacgctaccaatatgtttgccacc

SEQ ID NO: 20 - DNA, 36 bp, Artificial Sequence
  Description of Artificial Sequence: Synthetic primer
gcttcatcctactttgccaatatgtttgccacctgg

SEQ ID NO: 21 - DNA, 45 bp, Artificial Sequence
  Description of Artificial Sequence: Synthetic primer
cagattactgcttcatcctactttaccgctatgtttgccacctgg

SEQ ID NO: 22 - DNA, 42 bp, Artificial Sequence
  Description of Artificial Sequence: Synthetic primer
cttcatcctactttaccaatgcgtttgccacctggtctcctt

SEQ ID NO: 23 - DNA, 40 bp, Artificial Sequence
  Description of Artificial Sequence: Synthetic primer
cttcatcctactttaccaatatggctgccacctggtctcc

SEQ ID NO: 24 - DNA, 43 bp, Artificial Sequence
  Description of Artificial Sequence: Synthetic primer
cctactttaccaatatgtttgccgcctggtctccttcaaaagc

SEQ ID NO: 25 - DNA, 42 bp, Artificial Sequence
  Description of Artificial Sequence: Synthetic primer
gccacctggtctcctgcaaaagctcgacttcacctccaaggg

SEQ ID NO: 26 - DNA, 35 bp, Artificial Sequence
  Description of Artificial Sequence: Synthetic primer
ggtctccttcagcagctcgacttcacctccaaggg

SEQ ID NO: 27 - DNA, 42 bp, Artificial Sequence
  Description of Artificial Sequence: Synthetic primer
ccttcaaaagctcgacttgccctccaagggaggagtaatgcc

SEQ ID NO: 28 - DNA, 42 bp, Artificial Sequence
  Description of Artificial Sequence: Synthetic primer
ccttcaaaagctcgacttcacgcccaagggaggagtaatgcc

SEQ ID NO: 29 - DNA, 42 bp, Artificial Sequence
  Description of Artificial Sequence: Synthetic primer
ccttcaaaagctcgacttcacctcgcagggaggagtaatgcc

SEQ ID NO: 30 - DNA, 38 bp, Artificial Sequence
  Description of Artificial Sequence: Synthetic primer
cgacttcacctccaaggggcgagtaatgcctggagacc

SEQ ID NO: 31 - DNA, 33 bp, Artificial Sequence
  Description of Artificial Sequence: Synthetic primer
ccaagggaggagtaatgcctgggcacctcaggt

SEQ ID NO: 32 - DNA, 44 bp, Artificial Sequence
  Description of Artificial Sequence: Synthetic primer
ggagtaatgcctggagacctgcggtgaataatccaaaagagtgg

SEQ ID NO: 33 - DNA, 36 bp, Artificial Sequence
  Description of Artificial Sequence: Synthetic primer
gcctggagacctcaggcgaataatccaaaagagtgg

SEQ ID NO: 34 - DNA, 45 bp, Artificial Sequence
  Description of Artificial Sequence: Synthetic primer
ggagtaatgcctggagacctcaggtggctaatccaaaagagtggc

SEQ ID NO: 35 - DNA, 39 bp, Artificial Sequence
  Description of Artificial Sequence: Synthetic primer
cctggagacctcaggtgaatgctccaaaagagtggctgc

SEQ ID NO: 36 - DNA, 39 bp, Artificial Sequence
  Description of Artificial Sequence: Synthetic primer
caggtgaataatccaaaagcgtggctgcaagtggacttc

SEQ ID NO: 37 - DNA, 40 bp, Artificial Sequence
  Description of Artificial Sequence: Synthetic primer
ggctgcaagtggacttcgcgaagacaatgaaagtcacagg

SEQ ID NO: 38 - DNA, 41 bp, Artificial Sequence
  Description of Artificial Sequence: Synthetic primer
ggacttccagaagacaatggcagtcacaggagtaactactc

SEQ ID NO: 39 - DNA, 45 bp, Artificial Sequence
  Description of Artificial Sequence: Synthetic primer
gtaactactcagggagtagcatctctgcttaccagcatgtatgtg

SEQ ID NO: 40 - DNA, 36 bp, Artificial Sequence
  Description of Artificial Sequence: Synthetic primer
cagggagtaaaagctctgcttaccagcatgtatgtg

SEQ ID NO: 41 - DNA, 45 bp, Artificial Sequence
  Description of Artificial Sequence: Synthetic primer
ctactcagggagtaaaatctgcgcttaccagcatgtatgtgaagg

SEQ ID NO: 42 - DNA, 41 bp, Artificial Sequence
  Description of Artificial Sequence: Synthetic primer
caggagtaaaatctctggctaccagcatgtatgtgaaggag

SEQ ID NO: 43 - DNA, 39 bp, Artificial Sequence
  Description of Artificial Sequence: Synthetic primer
ggagtaaaatctctgcttgccagcatgtatgtgaaggag

SEQ ID NO: 44 - DNA, 45 bp, Artificial Sequence
  Description of Artificial Sequence: Synthetic primer
ggagtaaaatctctgcttaccgccatgtatgtgaaggagttcctc

SEQ ID NO: 45 - DNA, 32 bp, Artificial Sequence
  Description of Artificial Sequence: Synthetic primer
gcatgtatgtgaaggcgttcctcatctccagc

SEQ ID NO: 46 - DNA, 36 bp, Artificial Sequence
  Description of Artificial Sequence: Synthetic primer
cctcatctccagcagtgcagatggccatcagtggac

SEQ ID NO: 47 - DNA, 37 bp, Artificial Sequence
  Description of Artificial Sequence: Synthetic primer
catctccagcagtcaagatggcgctcagtggactctc

SEQ ID NO: 48 - DNA, 44 bp, Artificial Sequence
  Description of Artificial Sequence: Synthetic primer
gcagtcaagatggccatgcgtggactctcttttttcagaatgcc

SEQ ID NO: 49 - DNA, 33 bp, Artificial Sequence
  Description of Artificial Sequence: Synthetic primer
ggccatcagtgggctctcttttttcagaatggc

SEQ ID NO: 50 - DNA, 45 bp, Artificial Sequence
  Description of Artificial Sequence: Synthetic primer
gatggccatcagtggactgccttttttcagaatggcaaagtaaag

SEQ ID NO: 51 - DNA, 43 bp, Artificial Sequence
  Description of Artificial Sequence: Synthetic primer
ggccatcagtggactctcttttttgcgaatggcaaagtaaagg

SEQ ID NO: 52 - DNA, 45 bp, Artificial Sequence
  Description of Artificial Sequence: Synthetic primer
cagtggactctcttttttcaggctggcaaagtaaaggtttttcag

SEQ ID NO: 53 - DNA, 46 bp, Artificial Sequence
  Description of Artificial Sequence: Synthetic primer
ggactctcttttttcagaatggcaaagcaaaggtttttcagaatgg

SEQ ID NO: 54 - DNA, 43 bp, Artificial Sequence
  Description of Artificial Sequence: Synthetic primer
cagaatggcaaagtaaaggcttttcagaatggcaaagtaaagg

SEQ ID NO: 55 - DNA, 43 bp, Artificial Sequence
  Description of Artificial Sequence: Synthetic primer
cagaatggcaaagtaaaggtttttgcgaatggcaaagtaaagg

SEQ ID NO: 56 - DNA, 45 bp, Artificial Sequence
  Description of Artificial Sequence: Synthetic primer
gtaaaggtttttcagggaaatgcagactccttcacacctgtggtg

SEQ ID NO: 57 - DNA, 38 bp, Artificial Sequence
  Description of Artificial Sequence: Synthetic primer
ggtttttcagggaaatcaagcctccttcacacctgtgg

SEQ ID NO: 58 - DNA, 40 bp, Artificial Sequence
  Description of Artificial Sequence: Synthetic primer
ggaaatcaagactccgccacacctgtggtgaactctctag

SEQ ID NO: 59 - DNA, 35 bp, Artificial Sequence
  Description of Artificial Sequence: Synthetic primer
ggaaatcaagactccttcgcacctgtggtgaactc

SEQ ID NO: 60 - DNA, 32 bp, Artificial Sequence
  Description of Artificial Sequence: Synthetic primer
ccttcacacctgtggcgaactctctagacccc

SEQ ID NO: 61 - DNA, 33 bp, Artificial Sequence
  Description of Artificial Sequence: Synthetic primer
cctgtggtgaacgctctagacccaccgttactg

SEQ ID NO: 62 - DNA, 38 bp, Artificial Sequence
  Description of Artificial Sequence: Synthetic primer
cctgtggtgaactctctagacgcaccgttactgactcg

SEQ ID NO: 63 - DNA, 37 bp, Artificial Sequence
  Description of Artificial Sequence: Synthetic primer
gacccaccgttagcgactcgctaccttcgaattcacc

SEQ ID NO: 64 - DNA, 36 bp, Artificial Sequence
  Description of Artificial Sequence: Synthetic primer
ctgactcgctaccttcaaattcacccccagagttgg

SEQ ID NO: 65 - DNA, 34 bp, Artificial Sequence
  Description of Artificial Sequence: Synthetic primer
cgctaccttcgaattgccccccagagttgggtgc

SEQ ID NO: 66 - DNA, 34 bp, Artificial Sequence
  Description of Artificial Sequence: Synthetic primer
ccttcgaattcaccccgcgagttgggtgcaccag

SEQ ID NO: 67 - DNA, 35 bp, Artificial Sequence
  Description of Artificial Sequence: Synthetic primer
ccagagttgggtggcccagattgccctgaggatgg

SEQ ID NO: 68 - DNA, 37 bp, Artificial Sequence
  Description of Artificial Sequence: Synthetic primer
ccccagagttgggtgcacgccattgccctgaggatgg

SEQ ID NO: 69 - DNA, 26 bp, Artificial Sequence
  Description of Artificial Sequence: Synthetic primer
ggttctgggctgcgcggcacaggacc

SEQ ID NO: 70 - DNA, 33 bp, Artificial Sequence
  Description of Artificial Sequence: Synthetic primer
ggttctgggctgcgaggcagcggacctctactg

SEQ ID NO: 71 - DNA, 45 bp, Artificial Sequence
  Description of Artificial Sequence: Synthetic primer
gctttactctccattcccaatggcatggcgcaactatttaaatcc

SEQ ID NO: 72 - DNA, 45 bp, Artificial Sequence
  Description of Artificial Sequence: Synthetic primer
gctttactctccattcccaatggcgcgctgcaactatttaaatcc

SEQ ID NO: 73 - DNA, 40 bp, Artificial Sequence
  Description of Artificial Sequence: Synthetic primer
gcatctgatattgctttactcgccattcccaatggcatgc

SEQ ID NO: 74 - DNA, 43 bp, Artificial Sequence
  Description of Artificial Sequence: Synthetic primer
gcatctgatattgctttagcctccattcccaatggcatgctgc

SEQ ID NO: 75 - DNA, 42 bp, Artificial Sequence
  Description of Artificial Sequence: Synthetic primer
gcagtaatctgtgcatctgctattgctttactctccattccc

SEQ ID NO: 76 - DNA, 42 bp, Artificial Sequence
  Description of Artificial Sequence: Synthetic primer
gcagtaatctgtgcagctgatattgctttactctccattccc

SEQ ID NO: 77 - DNA, 42 bp, Artificial Sequence
  Description of Artificial Sequence: Synthetic primer
ggtaaagtaggatgaagcagcaatctgtgcatctgatattgc

SEQ ID NO: 78 - DNA, 45 bp, Artificial Sequence
  Description of Artificial Sequence: Synthetic primer
ggtggcaaacatattggtaaaggcggatgaagcagtaatctgtgc

SEQ ID NO: 79 - DNA, 45 bp, Artificial Sequence
  Description of Artificial Sequence: Synthetic primer
ggtggcaaacatattggtagcgtaggatgaagcagtaatctgtgc

SEQ ID NO: 80 - DNA, 36 bp, Artificial Sequence
  Description of Artificial Sequence: Synthetic primer
ccaggtggcaaacatattggcaaagtaggatgaagc

SEQ ID NO: 81 - DNA, 45 bp, Artificial Sequence
  Description of Artificial Sequence: Synthetic primer
ccaggtggcaaacatagcggtaaagtaggatgaagcagtaatctg

SEQ ID NO: 82 - DNA, 42 bp, Artificial Sequence
  Description of Artificial Sequence: Synthetic primer
aaggagaccaggtggcaaacgcattggtaaagtaggatgaag

SEQ ID NO: 83 - DNA, 40 bp, Artificial Sequence
  Description of Artificial Sequence: Synthetic primer
ggagaccaggtggcagccatattggtaaagtaggatgaag

SEQ ID NO: 84 - DNA, 43 bp, Artificial Sequence
  Description of Artificial Sequence: Synthetic primer
gcttttgaaggagaccaggcggcaaacatattggtaaagtagg

SEQ ID NO: 85 - DNA, 42 bp, Artificial Sequence
  Description of Artificial Sequence: Synthetic primer
cccttggaggtgaagtcgagcttttgcaggagaccaggtggc

SEQ ID NO: 86 - DNA, 35 bp, Artificial Sequence
  Description of Artificial Sequence: Synthetic primer
cccttggaggtgaagtcgagctgctgaaggagacc

SEQ ID NO: 87 - DNA, 42 bp, Artificial Sequence
  Description of Artificial Sequence: Synthetic primer
ggcattactcctcccttggagggcaagtcgagcttttgaagg

SEQ ID NO: 88 - DNA, 42 bp, Artificial Sequence
  Description of Artificial Sequence: Synthetic primer
ggcattactcctcccttgggcgtgaagtcgagcttttgaagg

SEQ ID NO: 89 - DNA, 42 bp, Artificial Sequence
  Description of Artificial Sequence: Synthetic primer
ggcattactcctccctgcgaggtgaagtcgagcttttgaagg

SEQ ID NO: 90 - DNA, 38 bp, Artificial Sequence
  Description of Artificial Sequence: Synthetic primer
ggtctccaggcattactcgccccttggaggtgaagtcg

SEQ ID NO: 91 - DNA, 33 bp, Artificial Sequence
  Description of Artificial Sequence: Synthetic primer
acctgaggtgcccaggcattactcctcccttgg

SEQ ID NO: 92 - DNA, 44 bp, Artificial Sequence
  Description of Artificial Sequence: Synthetic primer
ccactcttttggattattcaccgcaggtctccaggcattactcc

SEQ ID NO: 93 - DNA, 36 bp, Artificial Sequence
  Description of Artificial Sequence: Synthetic primer
ccactcttttggattattcgcctgaggtctccaggc

SEQ ID NO: 94 - DNA, 45 bp, Artificial Sequence
  Description of Artificial Sequence: Synthetic primer
gccactcttttggattagccacctgaggtctccaggcattactcc

SEQ ID NO: 95 - DNA, 39 bp, Artificial Sequence
  Description of Artificial Sequence: Synthetic primer
gcagccactcttttggagcattcacctgaggtctccagg

SEQ ID NO: 96 - DNA, 39 bp, Artificial Sequence
  Description of Artificial Sequence: Synthetic primer
gaagtccacttgcagccacgcttttggattattcacctg

SEQ ID NO: 97 - DNA, 40 bp, Artificial Sequence
  Description of Artificial Sequence: Synthetic primer
cctgtgactttcattgtcttcgcgaagtccacttgcagcc

SEQ ID NO: 98 - DNA, 41 bp, Artificial Sequence
  Description of Artificial Sequence: Synthetic primer
gagtagttactcctgtgactgccattgtcttctggaagtcc

SEQ ID NO: 99 - DNA, 45 bp, Artificial Sequence
  Description of Artificial Sequence: Synthetic primer
cacatacatgctggtaagcagagatgctactccctgagtagttac

SEQ ID NO: 100 - DNA, 36 bp, Artificial Sequence
  Description of Artificial Sequence: Synthetic primer
cacatacatgctggtaagcagagcttttactccctg

SEQ ID NO: 101 - DNA, 45 bp, Artificial Sequence
  Description of Artificial Sequence: Synthetic primer
ccttcacatacatgctggtaagcgcagattttactccctgagtag

SEQ ID NO: 102 - DNA, 42 bp, Artificial Sequence
  Description of Artificial Sequence: Synthetic primer
ctccttcacatacatgctggtagccagagattttactccctg

SEQ ID NO: 103 - DNA, 39 bp, Artificial Sequence
  Description of Artificial Sequence: Synthetic primer
ctccttcacatacatgctggcaagcagagattttactcc

SEQ ID NO: 104 - DNA, 45 bp, Artificial Sequence
  Description of Artificial Sequence: Synthetic primer
gaggaactccttcacatacatggcggtaagcagagattttactcc

SEQ ID NO: 105 - DNA, 32 bp, Artificial Sequence
  Description of Artificial Sequence: Synthetic primer
gctggagatgaggaacgccttcacatacatgc

SEQ ID NO: 106 - DNA, 36 bp, Artificial Sequence
  Description of Artificial Sequence: Synthetic primer
gtccactgatggccatctgcactgctggagatgagg

SEQ ID NO: 107 - DNA, 37 bp, Artificial Sequence
  Description of Artificial Sequence: Synthetic primer
gagagtccactgagcgccatcttgactgctggagatg

SEQ ID NO: 108 - DNA, 44 bp, Artificial Sequence
  Description of Artificial Sequence: Synthetic primer
ggcattctgaaaaaagagagtccacgcatggccatcttgactgc

SEQ ID NO: 109 - DNA, 33 bp, Artificial Sequence
  Description of Artificial Sequence: Synthetic primer
gccattctgaaaaaagagagcccactgatggcc

SEQ ID NO: 110 - DNA, 45 bp, Artificial Sequence
  Description of Artificial Sequence: Synthetic primer
ctttactttgccattctgaaaaaaggcagtccactgatggccatc

SEQ ID NO: 111 - DNA, 43 bp, Artificial Sequence
  Description of Artificial Sequence: Synthetic primer
cctttactttgccattcgcaaaaaagagagtccactgatggcc

SEQ ID NO: 112 - DNA, 45 bp, Artificial Sequence
  Description of Artificial Sequence: Synthetic primer
ctgaaaaacctttactttgccagcctgaaaaaagagagtccactg

SEQ ID NO: 113 - DNA, 46 bp, Artificial Sequence
  Description of Artificial Sequence: Synthetic primer
ccattctgaaaaacctttgctttgccattctgaaaaaagagagtcc

SEQ ID NO: 114 - DNA, 43 bp, Artificial Sequence
  Description of Artificial Sequence: Synthetic primer
cctttactttgccattctgaaaagcctttacttagccattctg

SEQ ID NO: 115 - DNA, 43 bp, Artificial Sequence
  Description of Artificial Sequence: Synthetic primer
cctttactttgccattcgcaaaaacctttactttgccattctg

SEQ ID NO: 116 - DNA, 45 bp, Artificial Sequence
  Description of Artificial Sequence: Synthetic primer
caccacaggtgtgaaggagtctgcatttccctgaaaaacctttac

SEQ ID NO: 117 - DNA, 38 bp, Artificial Sequence
  Description of Artificial Sequence: Synthetic primer
ccacaggtgtgaaggaggcttcatttccctgaaaaacc

SEQ ID NO: 118 - DNA, 40 bp, Artificial Sequence
  Description of Artificial Sequence: Synthetic primer
ctagagagttcaccacaggtgtggcggagtcttgatttcc

SEQ ID NO: 119 - DNA, 35 bp, Artificial Sequence
  Description of Artificial Sequence: Synthetic primer
gagttcaccacaggtgcgaaggagtcttgatttcc

SEQ ID NO: 120 - DNA, 32 bp, Artificial Sequence
  Description of Artificial Sequence: Synthetic primer
ggggtctagagagttcgccacaggtgtgaagg

SEQ ID NO: 121 - DNA, 33 bp, Artificial Sequence
  Description of Artificial Sequence: Synthetic primer
cagtaacggtgggtctagagcgttcaccacagg

SEQ ID NO: 122 - DNA, 38 bp, Artificial Sequence
  Description of Artificial Sequence: Synthetic primer
cgagtcagtaacggtgcgtctagagagttcaccacagg

SEQ ID NO: 123 - DNA, 33 bp, Artificial Sequence
  Description of Artificial Sequence: Synthetic primer
cagtagaggtccgctgcctcgcagcccagaacc

SEQ ID NO: 124 - DNA, 36 bp, Artificial Sequence
  Description of Artificial Sequence: Synthetic primer
ccaactctgggggtgaatttgaaggtagcgagtcag

SEQ ID NO: 125 - DNA, 34 bp, Artificial Sequence
  Description of Artificial Sequence: Synthetic primer
gcacccaactctggggggcaattcgaaggtagcg

SEQ ID NO: 126 - DNA, 34 bp, Artificial Sequence
  Description of Artificial Sequence: Synthetic primer
ctggtgcacccaactcgcggggtgaattcgaagg

SEQ ID NO: 127 - DNA, 35 bp, Artificial Sequence
  Description of Artificial Sequence: Synthetic primer
ccatcctcagggcaatctgggccacccaactctgg

SEQ ID NO: 128 - DNA, 37 bp, Artificial Sequence
  Description of Artificial Sequence: Synthetic primer
ccatcctcagggcaatggcgtgcacccaactctgggg

SEQ ID NO: 129 - DNA, 26 bp, Artificial Sequence
  Description of Artificial Sequence: Synthetic primer
ggtcctgtgccgcgcagcccagaacc

SEQ ID NO: 130 - DNA, 33 bp, Artificial Sequence; identical to SEQ ID NO: 123
  Description of Artificial Sequence: Synthetic primer

(19) United States
(12) Patent Application Publication
(10) Pub. No.: US 2017/0051041 A1
(43) Pub. Date: Feb. 23, 2017

(54) FACTOR VIII B CELL EPITOPE VARIANTS HAVING REDUCED IMMUNOGENICITY

(71) Applicants: Kathleen PRATT, Bethesda, MD (US); Jason T. SCHUMAN, Seattle, WA (US); BLOODWORKS, Seattle, WA (US)

(72) Inventors: Kathleen PRATT, Bethesda, MD (US); Jason T. SCHUMAN, Lake Oswego, OR (US)

(21) Appl. No.: 15/119,106

(22) PCT Filed: Feb. 19, 2015
(86) PCT No.: PCT/US15/16692

§ 371 Date: Aug. 15, 2016

Related U.S. Application Data
(60) Provisional application No. 61/941,940, filed on Feb. 19, 2014.

(51) Int. Cl. C07K 14/755

(52) U.S. Cl. CPC C07K 14/755; A61K 38/00

(57) ABSTRACT

Provided herein are methods and compositions for preventing or reducing an initial immune response to factor VIII in patients suffering from hemophilia A and for reducing the intensity of the immune response in patients having pre-formed inhibitor antibodies against factor VIII.

### BACKGROUND

Factor VIII (FVIII) is a protein found in blood plasma which acts as a cofactor in the cascade of reactions leading to blood coagulation. A deficiency in the amount of FVIII activity in the blood results in the clotting disorder known as hemophilia A, which is primarily a congenital condition but can also be acquired in rare cases. Hemophilia A is currently treated with therapeutic preparations of FVIII derived from human plasma or manufactured using recombinant DNA technology. FVIII can be administered in response to a bleeding episode (on-demand therapy) and/or at frequent, regular intervals to prevent uncontrolled bleeding (prophylaxis).

Up to 30% of patients with severe hemophilia A (FVIII activity <1%) develop inhibitory antibodies to FVIII as a consequence of treatment with therapeutic preparations of FVIII (Lusher et al., J Thromb Haemost: 2:574-583 (2004); Scharrer et al., Haemophilia; 5:145-154 (1999)). Frequently, the inhibitors are persistent and of sufficiently high titer that infusion of FVIII concentrates is ineffective for controlling bleeding episodes. Inhibitor formation therefore represents a major obstacle in treating patients with hemophilia A. In patients with high titer inhibitors, acute bleeding can sometimes be controlled by infusion of bypass clotting factors, including activated prothrombin complex concentrates and/or recombinant human factor VIIa. Bypass factors are considerably more expensive than standard FVIII concentrates, and their use in long-term prophylaxis regimens is limited due to their thrombogenic potential and unreliable hemostatic profile (Hay et al., Br J Haematol; 133:591-605 (2006); Paisley et al., Haemophilia; 9:405-417 (2003)). As a result, patients with persistent high titer inhibitors have a markedly reduced quality of life due to frequent joint bleeds and the early progression of arthropathies (Morfini et al., Haemophilia; 13:606-612 (2007)).

Accordingly, there is a need in the art for safe, effective, and/or low cost treatments for hemophilia patients with inhibitors to FVIII. There is also a need for less immunogenic/antigenic hemophilia treatments, which would reduce and/or prevent the incidence of inhibitor development.

### SUMMARY

Disclosed herein is a modified Factor VIII polypeptide comprising at least one amino acid modification in an unmodified Factor VIII polypeptide, wherein the at least one amino acid modification is at a position corresponding to positions 2173-2332 of the C2 domain of the amino acid sequence set forth in SEQ ID NO: 1, and wherein the at least one amino acid modification is at a position corresponding to position D2187, K2207, H2211, L2212, Q2213, E2181, T2202, S2206, R2220, E2181, S2206, F2196, N2198, F2200, T2202, S2250, L2251, L2252, T2253, S2254, H2315, M2199, R2215, Q2270, Q2316, F2196, N2198, F2200, T2202, N2225, E2228, L2252, S2254, Q2316, T2197, Q2222, K2239, H2315, Y2195, M2199, N2224, K2249, S2250, L2251, T2253, H2309, N2225, E2228, L2273, R2307, H2309, T2197, Q2270, R2220, K2239, H2269, V2280, R2220, T2272, L2273, V2282, H2309, H2269, Q2270, V2280, Q2311, or R2307 of the amino acid sequence set forth in SEQ ID NO:1.

In some embodiments, the at least one amino acid modification is at a position corresponding to position D2187, K2207, H2211, L2212, Q2213, E2181, T2202, S2206, R2220, E2181, or S2206 of the amino acid sequence set forth in SEQ ID NO:1.

In some embodiments, the at least one amino acid modification is at a position corresponding to position E2181, D2187, K2207, Q2213, S2206, or Q2213 of the amino acid sequence set forth in SEQ ID NO: 1.

In some embodiments, the at least one amino acid modification is at a position corresponding to position F2196, N2198, F2200, R2220, T2202, S2250, L2251, L2252, T2253, S2254 and H2315, E2181, M2199, R2215, Q2270, or Q2316 of the amino acid sequence set forth in SEQ ID NO:1.

In some embodiments, the at least one amino acid modification is at a position corresponding to position F2196, N2198, M2199, F2200, R2215, R2220, S2250, L2252, or S2254 of the amino acid sequence set forth in SEQ ID NO: 1.

In some embodiments, the at least one amino acid modification is at a position corresponding to position F2196, N2198, F2200, T2202, R2220, N2225, E2228, L2252, S2254, Q2316, T2197, Q2222, K2239, H2315, Y2195, M2199, N2224, K2249, S2250, L2251, T2253, or H2309 of the amino acid sequence set forth in SEQ ID NO:1.

In some embodiments, the at least one amino acid modification is at a position corresponding to position F2196, N2198, T2202, R2220, Q2222, N2224, N2225, E2228, K2239, L2251, L2252, T2253, S2254, H2315, or Q2316 of the amino acid sequence set forth in SEQ ID NO:1.

In some embodiments, the at least one amino acid modification is at a position corresponding to position N2225, E2228, L2273, R2307, H2309, T2197, Q2270, R2220, K2239, H2269, or V2280 of the amino acid sequence set forth in SEQ ID NO:1.

In some embodiments, the at least one amino acid modification is at a position corresponding to position L2273, E2228, L2273, and R2307 of the amino acid sequence set forth in SEQ ID NO: 1.

In some embodiments, the at least one amino acid modification is at a position corresponding to position R2220, T2272, L2273, V2282, H2309, H2269, Q2270, V2280, Q2311, or R2307 of the amino acid sequence set forth in SEQ ID NO:1.

In some embodiments, the at least one amino acid modification is at a position corresponding to position Q2270, L2273, R2307, L2273, and V2280 of the amino acid sequence set forth in SEQ ID NO:1.

In some embodiments, the at least one amino acid modification is an amino acid substitution at a position of the amino acid sequence set forth in SEQ ID NO: 1, selected from the group consisting of E2181A, D2187A, Y2195A, F2196A, T2197A, N2198A, M2199A, F2200A, T2202A, S2206A, K2207A, H2211A, L2212A, Q2213A, R2215A, R2220A, Q2222A, N2224A, N2225A, E2228A, K2239A, K2249A, S2250A, L2251A, L2252A, T2253A, S2254A, H2269A, Q2270A, T2272A, L2273A, V2280A, V2282A, R2307Q, H2309A, Q2311A, H2315A, and Q2316A.

In some embodiments, the at least one amino acid modification is an amino acid substitution at a position of the amino acid sequence set forth in SEQ ID NO: 1, selected from F2196K or F2196A.

In some embodiments, the at least one amino acid modification is an amino acid deletion. In some embodiments, the at least one amino acid modification is an amino acid addition. In some embodiments, the at least one amino acid modification is an amino acid substitution. In some embodiments, the at least one amino acid modification is a covalent chemical modification.

In some embodiments, the at least one amino acid modification is a modification in a B cell epitope.

In some embodiments, the modified Factor VIII polypeptide retains an activity of the unmodified Factor VIII polypeptide. In some embodiments, the modified Factor VIII polypeptide exhibits reduced immunogenicity/antigenicity upon administration to a subject compared to the unmodified Factor VIII polypeptide.

In some embodiments, the unmodified Factor VIII polypeptide comprises an amino acid sequence that has 40%, 50%, 60%, 70%, 80%, 90%, 91%, 92%, 93%, 94%, 95%, 96%, 97%, 98%, or 99% sequence identity to the amino acid sequence set forth in SEQ ID NO:1, excluding amino acid modification(s).

In some embodiments, the modified Factor VIII polypeptide is a human polypeptide. In some embodiments, the modified Factor VIII polypeptide is a non-human polypeptide.

In some embodiments, the modified Factor VIII polypeptide comprises at least 2, 3, 4, 5, 6, 7, 8, 9, 10, 11, 12, 13, 14, 15, 16, 17, 18, 19, or 20 amino acid modifications.

In some embodiments, the modified Factor VIII polypeptide comprises 6 amino acid modifications.

In some embodiments, the modified Factor VIII polypeptide comprises a single amino acid modification.

In some embodiments, the modified Factor VIII polypeptide further comprises at least one additional amino acid modification.

In some embodiments, the at least one additional amino acid modification is a modification in a T cell epitope.

In some embodiments, the at least one additional amino acid modification is at a position corresponding to positions 2173-2332 of the C2 domain of the amino acid sequence set forth in SEQ ID NO:1 or positions 373-740 of the A2 domain of the amino acid sequence set forth in SEQ ID NO:1.

In some embodiments, the at least one additional amino acid modification is at a position corresponding to positions 2194-2213 of the amino acid sequence set forth in SEQ ID NO: 1. In some embodiments, the at least one additional amino acid modification is at a position corresponding to positions 2202-2221 of the amino acid sequence set forth in SEQ ID NO: 1. In some embodiments, the at least one additional amino acid modification is at a position corresponding to positions 2194-2205 of the amino acid sequence set forth in SEQ ID NO: 1. In some embodiments, the at least one additional amino acid modification is at a position corresponding to positions 2196-2204 of the amino acid sequence set forth in SEQ ID NO: 1. In some embodiments, the at least one additional amino acid modification is at a position corresponding to position F2196, M2199, A2201, or S2204 of the amino acid sequence set forth in SEQ ID NO:1.

Also disclosed herein is a pharmaceutical composition comprising a modified Factor VIII polypeptide as described herein, and a pharmaceutically acceptable excipient.

Also disclosed herein is a nucleic acid molecule encoding the modified Factor VIII polypeptide as described herein.

Also disclosed herein is a recombinant expression vector comprising a nucleic acid molecule as described herein. Also disclosed herein is a host cell transformed with the recombinant expression vector.

Also disclosed herein is a method of making a modified Factor VIII polypeptide as disclosed herein, comprising: providing a host cell comprising a nucleic acid sequence that encodes the modified Factor VIII polypeptide; and maintaining the host cell under conditions in which the modified Factor VIII polypeptide is expressed.

Also disclosed herein is a method for reducing or preventing a condition associated with an immune response to Factor VIII, comprising administering to a subject in need thereof an effective amount of the modified Factor VIII polypeptide disclosed herein.

In some embodiments, the condition is the formation of an inhibitor antibody against Factor VIII. In some embodiments, the immune response is an initial immune response. In some embodiments, the subject is a naïve subject, e.g., a subject who has never been treated with Factor VIII. In some embodiments, the subject has been infused with Factor VIII but has not developed an inhibitor antibody response requiring additional treatment for bleeding in addition to or instead of Factor VIII infusions.

Also disclosed herein is a method for treating or reducing a condition associated with an immune response to Factor VIII, comprising administering to a subject in need thereof an effective amount of the modified Factor VIII polypeptide disclosed herein.

In some embodiments, the condition is the presence of an inhibitor antibody against Factor VIII. In some embodiments, the condition is the presence of a pre-formed inhibitor antibody against Factor VIII. In some embodiments, the method reduces the intensity of the condition.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1. Representative superimposed sensorgrams showing single-cycle kinetics experiments in which WT-FVIII-C2 and FVIII-C2 muteins were injected at five increasing concentrations over biosensor flow channels with captured murine anti-FVIII mAbs, as indicated. Residues were flagged as potential contributors to the epitope if the k_{d} for the FVIII-C2 mutein was >2.0× the k_{d} for the wild-type protein. A. Alanine substitutions at residues E2228, L2252, S2254, H2315 and Q2316 met this criterion in this SPR run with mAb 1B5 (which is a type B inhibitor). Separate SPR runs identified residues F2196, T2197, N2198, F2200, T2202, R2220, Q2222, N2225 and K2239 (subsequently identified as an outlier) as also possibly contributing to the epitope recognized by mAb 1B5. B. WT-FVIII-C2 and FVIII-C2 muteins Y2195A, Q2213A, N2225A, Q2270A and T2272A were injected over the flow channel containing mAb I54 (which is a type A inhibitor). The substitution Q2213A abrogated binding to this mAb, indicating that Q2213 forms part of the epitope recognized by I54. C. WT-FVIII-C2 and FVIII-C2 muteins Y2195A, Q2213A, N2225A, Q2270A and T2272A were injected over the flow channel in a second SPR run to analyze interactions of FVIII-C2 muteins with mAb 1B5. Altered binding kinetics indicated that residue N2225 forms part of the epitope recognized by 1B5. D. WT-FVIII-C2 and FVIII-C2 muteins Y2195A, Q2213A, N2225A, Q2270A and T2272A were injected over the flow channel containing mAb 2-117 (which is a type C inhibitor). Altered binding kinetics indicated that residues Q2270 and T2272 form part of the epitope recognized by 2-117.

FIG. 2. The B-cell epitopes indicated by the SPR experiments are visualized using space-filling depictions of the FVIII-C2 domain crystal structure in standard orientation, with the membrane-interacting loops pointing downwards. The FVIII-C2 structure is also shown rotated 180 degrees about the vertical axis for Type AB and Type B mAbs, in order to visualize both sides of the molecule. The B-cell epitopes identified on the basis of altered binding kinetics are color-coded according to FVIII inhibitor type, i.e. A (red/salmon), AB (orange/yellow). B (dark/light green), BC (dark/light blue) and C (dark/light magenta). The darker colors indicate residues for which amino acid substitutions increased the residence time by at least 10× compared to that for WT-FVIII-C2 binding to this mAb. Substitutions abrogating binding were also colored darker. Substitutions for which accurate k_{d} values could not be obtained were not colored darker, because their effects on kinetics may have been due in part to effects on protein stability. Several “outlier” residues identified as candidates using the cutoff criterion of k_{d}(mutein)>2.0 k_{d}(WT) are not shown, as they were eliminated following visualization of the FVIII-C2 crystal structure.

FIG. 3. Visualization of FVIII-C2 epitopes in the B-domain-deleted FVIII crystal structure²¹. With the exception of Type A inhibitors, the neutralizing mAbs analyzed herein bound to an outside-facing surface of FVIII, where they would not be expected to interfere with the packing or orientation of FVIII domains. A. The type BC and C epitopes recognized by non-classical inhibitors are shown as space-filling dark purple spheres in the FVIII structure²¹. The protein is oriented with the membrane binding residues M2199, F2200, L2251, L2252, K2092 and F2093 pointing down. Also indicated by space filling spheres are residues known to be at the interface between FVIIIa and activated factor IX (FIXa) in the intrinsic tenase complex: region i (FVIII residues 558-565) is colored light blue; region ii consists of residues near residue 712, which is colored purple; region iii (residues 1811-1818) is colored salmon. Note that the Type BC epitope, which corresponds to a docking site for activated thrombin, is on the opposite side of FVIII to the FVIIIa-FIXa interface. B. All of the residues identified as contributing to each of the five types of epitopes are shown as space-filling spheres in the FVIII crystal structure²¹. C. Venn diagram depictions of specific amino acid residues localized to the B-cell epitopes A, AB, B, BC and C. Amino acid side chains that SPR assays, followed by visual inspection of the FVIII-C2 crystal structure, indicated contribute to functional B-cell epitopes in human FVIII. Amino acid residues in porcine FVIII that differ from the human FVIII sequence at positions corresponding to functional B-cell epitopes identified in this study are indicated in the second Venn diagram.

FIG. 4. SDS-PAGE showing representative purification and characterization steps for FVIII-C2 muteins Y2195A, M2199A and T2197A. L=E. coli lysate (starting material for the purification); PT=pooled pass-through fractions from the Ni-NTA His-bind column; P=purified protein following the endotoxin removal step.

### DETAILED DESCRIPTION

The development of neutralizing anti-factor VIII (FVIII) antibodies is a serious complication that may be encountered when FVIII replacement therapy is administered to hemophilia A (HA) patients, affecting 25-30% of the treated HA population with a peak occurrence following ˜14 FVIII infusions¹⁻³. Autoimmune responses to FVIII can also occur⁴, and although this happens only rarely, the resulting bleeding phenotype can be severe. Inhibitors can be difficult and extremely expensive to manage clinically. Interestingly, porcine FVIII has been used effectively in the clinic as a “bypass” therapy, i.e. a therapeutic protein that can evade neutralization by anti-FVIII antibodies in many allo- and autoimmune inhibitor patients⁵⁻⁷. However, some patients have or could develop antibodies that neutralize porcine FVIII as well⁸, due to antigenic cross-reactivity⁹ or because regions in which the porcine sequence differs from the human FVIII sequence stimulate effector T cells, leading to antibody production. Identification of the binding sites (B-cell epitopes) on FVIII that are recognized by inhibitors would allow rational design of novel therapeutic FVIII proteins that are more similar to human FVIII and hence likely to be less immunogenic.

The most common epitopes recognized by hemophilic inhibitors are on the FVIII A2 and C2 domains^{10,11}. The FVIII C2 domain (FVIII-C2) mediates numerous functions that are essential for the full procoagulant cofactor activity of FVIII, including membrane binding and assembly of the intrinsic tenase complex¹². The goal of the present study is to identify B-cell epitopes on FVIII-C2 that are recognized by neutralizing anti-FVIII antibodies. In an earlier study¹³, competition ELISA assays were employed to characterize 56 murine monoclonal antibodies (mAbs) that bound to FVIII-C2 and blocked FVIII procoagulant activity. Results of these assays indicated there were three distinct epitopes on this domain, types A, B and C, and inhibitory antibodies also bound to partially overlapping epitopes AB and BC. A, B and AB antibodies, termed “classical” anti-C2 antibodies, inhibit the assembly of the intrinsic tenase complex on negatively-charged phospholipid membranes. C and BC antibodies, termed “non-classical” anti-C2 antibodies, inhibit the proteolytic activation of FVIII to FVIIIa by thrombin and/or by activated factor X (FXa). In order to identify the specific amino acid residues comprising these five types of epitopes, 60 recombinant FVIII-C2 mutant proteins (muteins) plus the wild-type (WT) protein (WT-FVIII-C2) were generated using an E. coli expression system, 59 with an alanine substitution at a surface-exposed amino acid side chain plus the conservative substitution R2307Q. (The “legacy” numbering for FVIII residues is employed herein, for consistency with the earlier studies¹³). Surface plasmon resonance (SPR) experiments were carried out to measure binding kinetics of WT-FVIII-C2 and FVIII-C2 muteins to 10 representative mAbs from the series characterized earlier by competition ELISA and functional assays, as well as to the human-derived monoclonal anti-FVIII antibody BO2C11¹⁴.

Terms used in the claims and specification are defined as set forth below unless otherwise specified. In the case of direct conflict with a term used in a parent provisional patent application, the term used in the instant specification shall control.

As used herein, a “Factor VIII” (FVIII) refers to any factor VIII polypeptide or nucleotide, including but not limited to, a recombinantly produced polypeptide, a synthetically produced polypeptide and a factor VIII polypeptide extracted or isolated from cells or tissues including, but not limited to, liver and blood. Factor VIII includes related polypeptides from different species including, but not limited to animals of human and non-human origin. Human factor VIII includes factor VIII, allelic variant isoforms, synthetic molecules from nucleic acids, protein isolated from human tissue and cells, and modified forms thereof. Exemplary unmodified human factor VIII polypeptides include, but are not limited to, unmodified and wild-type native factor VIII polypeptide and the unmodified and wild-type precursor factor VIII polypeptide. The factor VIII polypeptides provided herein can be modified, such as by amino acid addition, amino acid substitution, amino acid deletion, or chemical modification or post-translational modification. Such modifications include, but are not limited to, covalent modifications, pegylation, albumination, glycosylation, farnysylation, carboxylation, hydroxylation, phosphorylation, and other polypeptide modifications known in the art.

Factor VIII includes factor VIII from any species, including human and non-human species. Factor VIII of non-human origin include, but are not limited to, murine, canine, feline, leporine, avian, bovine, ovine, porcine, equine, piscine, ranine, and other primate factor VIII.

Human and non-human factor VIII polypeptides include factor VIII polypeptides, allelic variant isoforms, tissue-specific isoforms and allelic variants thereof, synthetic molecules prepared by translation of nucleic acids, proteins isolated from human and non-human tissue and cells, chimeric factor VIII polypeptides and modified forms thereof. Human and non-human factor VIII also include fragments or portions of factor VIII that are of sufficient length or include appropriate regions to retain at least one activity of the full-length mature polypeptide. Human and non-human factor VIII polypeptides also can include factor VIII polypeptides that are of sufficient length to inhibit one or more activities of a full-length mature factor VIII polypeptide.

As used herein, an “active portion or fragment of a factor VIII polypeptide” refers to a portion of a human or non-human factor VIII polypeptide that includes at least one modification provided herein and exhibits an activity, such as one or more activities of a full-length factor VIII polypeptide or possesses another activity. Activity can be any percentage of activity (more or less) of the full-length polypeptide, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more activity compared to the full polypeptide. Assays to determine function or activity of modified forms of factor VIII include those known to those of skill in the art, and exemplary assays are included herein. Activity also includes activities possessed by a fragment or modified form that are not possessed by the full length polypeptide or unmodified polypeptide.

As used herein, “native factor VIII” refers to a factor VIII polypeptide encoded by a naturally occurring factor VIII gene that is present in an organism in nature, including a human or other animal. Included among native factor VIII polypeptides are the encoded precursor polypeptide, fragments thereof, and processed forms thereof, such as any pre- or post-translationally processed or modified form thereof.

As used herein. “unmodified protein,” “unmodified polypeptide,” “unmodified target protein,” “unmodified factor VIII” and grammatical variations thereof refer to a starting polypeptide that is selected for modification as provided herein. The starting target polypeptide can be a naturally-occurring, wild-type form of a polypeptide. In addition, the starting target polypeptide can be altered or mutated, such that it differs from a native wild type isoform but is nonetheless referred to herein as a starting unmodified target protein relative to the subsequently modified polypeptides produced herein. Thus, existing proteins known in the art that have been modified to have a desired increase or decrease in a particular activity or property compared to an unmodified reference protein can be selected and used as the starting unmodified target protein. For example, a protein that has been modified from its native form by one or more single amino acid changes and possesses either an increase or decrease in a desired property, such as a change in an amino acid residue or residues to alter glycosylation, or to alter half-life, etc., can be a target protein, referred to herein as unmodified, for further modification of either the same or a different property.

Existing proteins known in the art that previously have been modified to have a desired alteration, such as an increase or decrease, in a particular biological activity or property compared to an unmodified or reference protein can be selected and used as provided herein for identification of structurally homologous loci on other structurally homologous target proteins. For example, a protein that has been modified by one or more single amino acid changes and possesses either an increase or decrease in a desired property or activity, such as for example reduced immunogenicity/antigenicity, can be utilized with the methods provided herein to identify on structurally homologous target proteins, corresponding structurally homologous loci that can be replaced with suitable replacing amino acids and tested for either an increase or decrease in the desired activity.

As used herein, an “activity” or a “functional activity” of a factor VIII polypeptide refers to any activity exhibited by a factor VIII polypeptide. Activities of a factor VIII polypeptide can be tested in vitro and/or in vivo and include, but are not limited to, coagulation activity, anticoagulation activity, enzymatic activity, and peptidase activity. Activity can be assessed in vitro or in vivo using recognized assays. The results of such assays that indicate that a polypeptide exhibits an activity can be correlated to activity of the polypeptide in vivo, in which in vivo activity can be referred to as biological activity. Activity can be any level of percentage of activity of the polypeptide, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more of activity compared to the full polypeptide. Assays to determine functionality or activity of modified forms of factor VIII are known to those of skill in the art.

As used herein, “exhibits at least one activity” or “retains at least one activity” refers to the activity exhibited by a modified factor VIII polypeptide as compared to an unmodified factor VIII polypeptide of the same form and under the same conditions. For example, a modified factor VIII polypeptide is compared with an unmodified factor VIII polypeptide, under the same experimental conditions, where the only difference between the two polypeptides is the modification under study. Generally, a modified factor VIII polypeptide that retains an activity of an unmodified factor VIII polypeptide either improves or maintains the requisite biological activity of an unmodified factor VIII polypeptide. In some instances, a modified factor VIII polypeptide can retain an activity that is increased compared to an unmodified factor VIII polypeptide. In some cases, a modified factor VIII polypeptide can retain an activity that is decreased compared to an unmodified factor VIII polypeptide.

Activity of a modified factor VIII polypeptide can be any level of percentage of activity of the unmodified polypeptide, including but not limited to, 1% of the activity, 2%, 3%, 4%, 5%, 10%, 20%, 30%, 40%, 50%, 60%, 70%, 800%, 90%, 95%, 96%, 97%, 98%, 99%, 100%, 200%, 300%, 400%, 500%, or more activity compared to the unmodified polypeptide. For example, a modified factor VIII polypeptide retains at least about or 1%, 2%, 3%, 4%, 5%, 6%, 7%, 8%, 9%, 10%, 20%, 30%, 40%, 50/o, 60%, 70/a, 80%, 90%, 95%, 96%, 97%, 98% or at least 99% of the activity of the wild-type factor VIII polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified factor VIII.

As used herein, a “property” of a factor VIII polypeptide refers to any property exhibited by a factor VIII polypeptide. Changes in properties can alter an “activity” of the polypeptide. One example of a property of a modified factor VIII polypeptide is reduced immunogenicity/antigenicity.

As used herein, “factor VIII-associated disease or disorder” refers to any disease or disorder in which treatment with a factor VIII (e.g., modified factor VIII) ameliorates any symptom or manifestation of the disease or disorder. Exemplary factor VIII-associated diseases and disorders include, but are not limited to, hemorrhagic disorders, such as hemophilia. Accordingly, a disease or condition that is treated by administration of factor VIII includes any disease or condition for which factor VIII (e.g., modified factor VIII) is employed for treatment, including, but not limited to, hemorrhagic disorders, such as hemophilia.

As used herein, “hemophilia” refers to a bleeding disorder caused by or involving a deficiency in blood clotting factors. Hemophilia can be the result, for example, of absence, reduced expression, or reduced function of a clotting factor. The most common type of hemophilia is hemophilia A, which results from a deficiency in factor VIII. The second most common type of hemophilia is hemophilia B, which results from a deficiency in factor IX. Another, more rare form of hemophilia is hemophilia C, which results from a deficiency in factor XI. As used herein, “congenital hemophilia” refers to types of hemophilia that are inherited. Congenital hemophilia results from mutation, deletion, insertion, or other modification of a clotting factor gene in which the production of the clotting factor is absent, reduced, or non-functional. For example, hereditary mutations in clotting factor genes, such as factor VIII and factor IX result in the congenital hemophilias, Hemophilia A and B, respectively.

As used herein, “subject” to be treated includes humans and human or non-human animals. Mammals include, primates, such as humans, chimpanzees, gorillas and monkeys; domesticated animals, such as dogs, horses, cats, pigs, goats, cows, and rodents, such as mice, rats, hamsters and gerbils. As used herein, a patient is a human subject.

An “epitope” is a set of amino acids on a protein that are involved in an immunological response, such as antibody binding, class II binding, or T-cell activation. “Epitope” includes T cell epitopes and B cell epitopes.

An “epitope area” is defined as the amino acids situated close to the epitope sequence amino acids. Preferably, the amino acids of an epitope area are located <5 angstrom (ANG) from the epitope sequence. Hence, an epitope area also includes the corresponding epitope sequence itself. Modifications of amino acids of the “epitope area” can, in some embodiments, affect the immunogenic function of the corresponding epitope.

By the term “epitope sequence” is meant the amino acid residues of a parent protein, which have been identified to belong to an epitope by the methods of the present invention.

As used herein, “variant,” “factor VIII variant,” “modified factor VIII polypeptides” and “modified factor VIII” refers to a factor VIII that has one or more mutations or modifications (e.g., chemical conjugations, additions, substitutions, deletions) compared to an unmodified factor VIII. The one or more mutations can be one or amino acid replacements, insertions or deletions and any combination thereof. Typically, a modified factor VIII has one or more modifications in its primary sequence compared to an unmodified factor VIII polypeptide. For example, a modified factor VIII provided herein can have 1, 2, 3, 4, 5, 6, 7, 8, 9, 10, 11, 12, 13, 14, 15, 16, 17, 18, 19, 20, or more mutations compared to an unmodified factor VIII. Modifications that confer a property (such as, reduced immunogenicity/antigenicity) by virtue of a change in a primary amino acid sequence do not always require a change in post-translational modification of the modified polypeptide to confer the property. Any length polypeptide is contemplated as long as the resulting polypeptide exhibits at least one factor VIII activity associated with a native factor VIII polypeptide or inhibits at least one factor VIII activity associated with a native factor VIII polypeptide.

As used herein, a “single amino acid replacement” refers to the replacement of one amino acid by another amino acid. The replacement can be by a natural amino acid or non-natural amino acids. When one amino acid is replaced by another amino acid in a protein, the total number of amino acids in the protein is unchanged.

As used herein, the phrase “only one amino acid replacement occurs on each target protein” refers to the modification of a target protein, such that it differs from the unmodified form of the target protein by a single amino acid change. For example, in one embodiment, mutagenesis is performed by the replacement of a single amino acid residue at only one target position on the protein backbone, such that each individual mutant generated is the single product of each single mutagenesis reaction. The single amino acid replacement mutagenesis reactions are repeated for each of the replacing amino acids selected at each of the target positions. Thus, a plurality of mutant protein molecules are produced, whereby each mutant protein contains a single amino acid replacement at only one of the target positions.

As used herein, “at a position or positions corresponding to an amino acid position” or “at a position or positions corresponding to position or positions” of a protein or grammatical variations thereof, refers to amino acid positions that are determined to correspond to one another based on sequence and/or structural alignments with a specified reference protein. For example, in a position corresponding to an amino acid position of human factor VIII can be determined empirically by aligning the sequence of amino acids of human factor VIII with a particular factor VIII polypeptide of interest. Corresponding positions can be determined by such alignment by one of skill in the art using manual alignments or by using the numerous alignment programs available (for example, BLASTP). Corresponding positions also can be based on structural alignments, for example by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm.

As used herein, “at a position corresponding to” refers to a position of interest (i.e., base number or residue number) in a nucleic acid molecule or protein relative to the position in another reference nucleic acid molecule or protein. The position of interest to the position in another reference protein can be in, for example, a precursor protein, an allelic variant, a heterologous protein, an amino acid sequence from the same protein of another species, etc. Corresponding positions can be determined by comparing and aligning sequences to maximize the number of matching nucleotides or residues, for example, such that identity between the sequences is greater than 95%, 96%, 97%, 98% or 99% or more. The position of interest is then given the number assigned in the reference nucleic acid molecule.

As used herein, the terms “homology” and “identity” are used interchangeably, but homology for proteins can include conservative amino acid changes. In general to identify corresponding positions the sequences of amino acids are aligned so that the highest order match is obtained (see, such as: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80%, or 90% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology vary). Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85: 2444 (other programs include the GCG program package (Devereux, J., et al. (1984) Nucleic Acids Research 12(1): 387), BLASTP, BLASTN, FASTA (Atschul, S. F., et al. (1990) J. Molec. Biol. 215:403; Guide to Huge Computers, Martin J. Bishop, ed., Academic Press, San Diego (1994), and Carillo et al. (1988) SIAM J Applied Math 48: 1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (such as, Needleman et al. (1970) J. Mol. Biol. 48: 443, as revised by Smith and Waterman (1981) Adv. Appl. Math. 2: 482. Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. (1986) Nucl. Acids Res. 14: 6745, as described by Schwartz and Dayhoff, eds. (1979) Atlas of Protein Sequence and Structure, National Biomedical Research Foundation, pp. 353-358; (2) a penalty of 3.0 for each gap and an additional 0.10 penalty for each symbol in each gap; and (3) no penalty for end gaps.

Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, such as, 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, the phrase “sequence-related proteins” refers to proteins that have at least 50%, at least 60%, at least 70%, at least 80%, at least 90%, or at least 95% amino acid sequence identity or homology with each other.

As used herein, families of non-related proteins or “sequence-non-related proteins” refer to proteins having less than 50%, less than 40%, less than 30%, less than 20% amino acid identity, or homology with each other.

As used herein, it also is understood that the terms “substantially identical” or “similar” varies with the context as understood by those skilled in the relevant art.

As used herein. “a naked polypeptide chain” refers to a polypeptide that is not post-translationally modified or otherwise chemically modified, but contains only covalently linked amino acids.

As used herein, the amino acids that occur in the various sequences of amino acids provided herein are identified according to their known, three-letter or one-letter abbreviations. The nucleotides which occur in the various nucleic acid fragments are designated with the standard single-letter designations used routinely in the art. As used herein, an “amino acid” is an organic compound containing an amino group and a carboxylic acid group. A polypeptide comprises two or more amino acids. For purposes herein, amino acids include the twenty naturally-occurring amino acids, non-natural amino acids, and amino acid analogs (i.e., amino acids wherein the α-carbon has a side chain). As used herein, the abbreviations for any protective groups, amino acids and other compounds are, unless indicated otherwise, in accord with their common usage, recognized abbreviations, or the IUPAC-IUB Commission on Biochemical Nomenclature (1972) Biochem. 11:1726). Each naturally occurring L-amino acid is identified by the standard three letter code (or single letter code) or the standard three letter code (or single letter code) with the prefactor VIII “L-;” the prefactor VIII “D-” indicates that the stercoisomeric form of the amino acid is D.

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. “NH2” refers to the free amino group present at the amino terminus of a polypeptide. “COOH” refers to the free carboxy group present at the carboxyl terminus of a polypeptide. In keeping with standard polypeptide nomenclature described in (1969) J. Biol. Chem., 243: 3552-3559, and adopted 37.C.F.R. 1.821-1.822.

All amino acid residue sequences represented herein by formulae have a left to right orientation in the conventional direction of amino-terminus to carboxyl-terminus. In addition, the phrase “amino acid residue” is broadly defined to include the amino acids listed herein and modified and unusual amino acids, such as those referred to in 37 C.F.R. 1.821-1.822, and incorporated herein by reference. Furthermore, a dash at the beginning or end of an amino acid residue sequence indicates a peptide bond to a further sequence of one or more amino acid residues, to an amino-terminal group such as NH2 or to a carboxyl-terminal group such as COOH.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-stereoisomers of amino acids. Exemplary non-natural amino acids are described herein and are known to those of skill in the art.

Conservative substitutions are the replacements of amino acids of one class with another member of that class. Examples of such conservative substitutions are: among the aliphatic amino acids Ala, Val, Leu, Ile and Met; interchange of the hydroxyl residues Ser and Thr, exchange of the acidic residues Asp and Glu, substitution between the amide residues Asn and Gin, exchange of the basic residues Lys and Arg and replacements among the aromatic residues Phe, Tyr, and Trp.

A non-conservative substitution is the replacement of an amino acid with an amino acid of dissimilar structure, such as one from a different class as described above. Examples of such substitutions include the substitution of a polar for a non-polar amino acid, a hydrophobic for a hydrophilic amino acid, a charged for a non-charged or oppositely charged amino acid, a bulky for a non-bulky side chain containing amino acid, or their converses, among other possible non-conservative substitutions.

As used herein, nucleic acids include DNA. RNA, and analogs thereof, including protein nucleic acids (PNA) and mixtures thereof. Nucleic acids can be single- or double-stranded. When referring to probes or primers (optionally labeled with a detectable label, such as, a fluorescent or a radiolabel), single-stranded molecules are contemplated. Such molecules are typically of a length such that they are statistically unique of low copy number (typically less than 5, generally less than 3) for probing or priming a library. Generally a probe or primer contains at least 10, 15, 20, 25, or 30 contiguous nucleic acid bases of sequence complementary to, or identical to, a gene of interest. Probes and primers can be 5, 6, 7, 8, 9, 10, or more, 20 or more, 30 or more, 50 or more, 100, or more nucleic acids long.

As used herein, heterologous or foreign nucleic acid, such as DNA and RNA, are used interchangeably and refer to DNA or RNA that does not occur naturally as part of the genome in which it occurs or is found at a locus or loci in a genome that differs from that in which it occurs in nature. Heterologous nucleic acid includes nucleic acid not endogenous to the cell into which it is introduced, but that has been obtained from another cell or prepared synthetically. Generally, although not necessarily, such nucleic acid encodes RNA and proteins that are not normally produced by the cell in which it is expressed. Heterologous DNA herein encompasses any DNA or RNA that one of skill in the art recognizes or considers as heterologous or foreign to the cell or locus in or at which it is expressed. Heterologous DNA and RNA also can encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins (such as, a protein that confers drug resistance), nucleic acid that encodes therapeutically effective substances (such as, anti-cancer agents), enzymes and hormones, and DNA that encodes other types of proteins (such as, antibodies). Hence, herein heterologous DNA or foreign DNA includes a DNA molecule not present in the exact orientation and position as the counterpart DNA molecule found in the genome. It also can refer to a DNA molecule from another organism or species (i.e., exogenous).

As used herein, “isolated with reference to a nucleic acid molecule or polypeptide or other biomolecule” means that the nucleic acid or polypeptide has separated from the genetic environment from which the polypeptide or nucleic acid were obtained. It also can mean altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been partially or substantially purified from a recombinant host cell or from a native source. For example, a recombinantly produced version of a compound can be substantially purified by the one-step method described in Smith et al. (1988) Gene, 67:31-40. The terms isolated and purified can be used interchangeably.

Thus, by “isolated” it is meant that the nucleic acid is free of coding sequences of those genes that, in the naturally-occurring genome of the organism (if any), immediately flank the gene encoding the nucleic acid of interest. Isolated DNA can be single-stranded or double-stranded, and can be genomic DNA, cDNA, recombinant hybrid DNA or synthetic DNA. It can be identical to a starting DNA sequence or can differ from such sequence by the deletion, addition, or substitution of one or more nucleotides.

“Purified” preparations made from biological cells or hosts mean at least the purity of a cell extracts containing the indicated DNA or protein including a crude extract of the DNA or protein of interest. For example, in the case of a protein, a purified preparation can be obtained following an individual technique or a series of preparative or biochemical techniques, and the DNA or protein of interest can be present at various degrees of purity in these preparations. The procedures can include, but are not limited to, ammonium sulfate fractionation, gel filtration, ion exchange chromatography, affinity chromatography, density gradient centrifugation, and electrophoresis.

A preparation of DNA or protein that is “substantially pure” or “isolated” refers to a preparation substantially free from naturally-occurring materials with which such DNA or protein is normally associated in nature and generally contains 5% or less of the other contaminants.

A cell extract that contains the DNA or protein of interest refers to a homogenate preparation or cell-free preparation obtained from cells that express the protein or contain the DNA of interest. The term “cell extract” is intended to include culture medium, especially spent culture medium from which the cells have been removed.

As used herein, “recombinant” refers to any progeny formed as the result of genetic engineering.

As used herein, the phrase “operatively linked” with reference to a nucleic acid molecule generally means the sequences or segments have been covalently joined into one piece of DNA, whether in single- or double-stranded form, whereby control or regulatory sequences on one segment control or permit expression or replication or other such control of other segments. The two segments are not necessarily contiguous. For gene expression, a DNA sequence and a regulatory sequence(s) are connected in such a way to control or permit gene expression when the appropriate molecular, such as, transcriptional activator proteins, are bound to the regulatory sequence(s).

As used herein, “production by recombinant means by using recombinant DNA methods” means the use of the well-known methods of molecular biology for expressing proteins encoded by cloned DNA, including cloning expression of genes and methods.

The term “ameliorating” refers to any therapeutically beneficial result in the treatment of a disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Factor VIII

Factor VIII (FVIII) exists naturally and in therapeutic preparations as a heterogeneous distribution of polypeptides arising from a single gene product (e.g., Andersson et al., Proc. Natl. Acad. Sci. USA, 83, 2979-2983 (1986), herein incorporated by reference). “Factor VIII” or “FVIII” refers to all such polypeptides, whether derived from blood plasma or produced through the use of recombinant DNA techniques or by other means.

FVIII is secreted as an approximately 300 kDa single chain glycoprotein having the following domain organization NH₂-A1-A2-B-A3-C1-C2-COOH, where each “domain” comprises a structural unit encoded by a continuous sequence of amino acids. FVIII isolated from plasma comprises two subunits, known as the heavy chain and light chain. The FVIII heavy chain comprises the A1, A2, and B domains, and the FVIII light chain comprises the A3, C1, and C2 domains. The B domain has no known biological function in clot formation and can be wholly or partially removed without significantly altering FVIII function.

FVIII generally circulates complexed with another plasma protein, von Willebrand factor (vWF), which is present in a large molar excess (˜50:1) to FVIII in plasma and protects FVIII from premature degradation by plasma proteases. FVIII is proteolytically activated primarily by thrombin (factor IIa), which cleaves the heavy chain between the A1 and A2 domains and dissociates FVIII from von Willebrand factor (vWF) to form factor VIIIa (FVIIIa), which is the active form of FVIII having coagulant activity. FVIIIa acts as a co-factor of activated Factor IX, which accelerates the activation of Factor X, which converts prothrombin into thrombin, which converts fibrinogen into fibrin, which induces clotting.

The human FVIII gene has been isolated and expressed in mammalian cells, as reported by various authors, including Wood et al. in Nature (1984) 312: 330-337 and the amino-acid sequence was deduced from cDNA. U.S. Pat. No. 4,965,199 discloses a recombinant DNA method for producing FVIII in mammalian host cells and purification of human FVIII. The human FVIII detailed structure has been extensively investigated. The cDNA nucleotide sequence encoding human FVIII and predicted amino-acid sequence have been disclosed for instance in U.S. Pat. No. 5,663,060, herein incorporated by reference. In some embodiments, FVIII is a nucleotide sequence encoding human FVIII and the corresponding amino acid sequence are shown in GenBank accession number NM_000132.2, herein incorporated by reference. In some embodiments, FVIII is a nucleotide sequence encoding human FVIII and the corresponding amino acid sequence are shown in GenBank accession number NM_000132.3, herein incorporated by reference. In some embodiments, FVIII is a nucleotide sequence encoding human FVIII with Asp1241 (e.g., Kogenate™) and the corresponding amino acid sequence. In some embodiments, FVIII is a nucleotide sequence encoding human FVIII with Glu1241 (e.g., Recombinate™) and the corresponding amino acid sequence.

Compositions

The present disclosure relates generally to methods and compositions for ameliorating or preventing the adverse effects of “inhibitor” antibodies in hemophilia patients. One aspect focuses on the mechanisms and structural determinants involved in initiating an inhibitor response. Inhibitor formation is T-cell dependent and involves recognition of specific epitopes on FVIII by antigen-specific T-cells. Factor VIII polypeptides are processed by antigen-presenting cells, which display factor VIII polypeptides to antigen-specific T-cells via cell surface HLA class II complexes. Antigen-specific T-cells recognize and bind certain peptide-HLA HI complexes, leading to T-cell activation and downstream stimulation of an antibody response. Disclosed herein are several T-cell epitopes identified using T-cells isolated from hemophilia A patients with inhibitors and characterization of the minimum structural features required for association with HLA II molecules and recognition by T-cells.

Contemplated herein are modified factor VIII polypeptides that differ from unmodified or wild-type factor VIII polypeptides with respect to a property or an activity. Modified factor VIII polypeptides provided herein can have reduced immunogenicity/antigenicity as compared to unmodified factor VIII polypeptides.

Provided herein are methods for reducing the immunogenicity/antigenicity of a factor VIII polypeptide. Provided herein are methods of modifying factor VIII polypeptides to reduce its immunogenicity/antigenicity. Provided herein are modified factor VIII polypeptides in which the primary amino acid sequence is modified to confer reduced immunogenicity/antigenicity. Among the amino acid modifications provided herein are such modifications including replacement of amino acids in the primary sequence of the factor VIII polypeptide in order to reduce the immunogenicity/antigenicity of the factor VIII polypeptide. Further modifications of the factor VIII polypeptide can be included, such as, but not limited to, addition of carbohydrate, phosphate, sulfur, hydroxyl, carboxyl, and polyethylene glycol (PEG) moieties. Thus, the modified factor VIII polypeptides provided herein can be modified, for example, by glycosylation, phosphorylation, sulfation, hydroxylation, carboxylation, and/or PEGylation. Such modifications can be performed in vivo or in vitro.

Provided herein are modified factor VIII polypeptides that display reduced immunogenicity/antigenicity. The reduced immunogenicity/antigenicity of the modified factor VIII polypeptide can be decreased by an amount that is at least about or 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the immunogenicity/antigenicity of the unmodified factor VIII polypeptide. In some examples, the reduced immunogenicity/antigenicity of the modified factor VIII polypeptide can be decreased by an amount that is at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times when compared to the immunogenicity/antigenicity of the unmodified factor VIII polypeptide. Hence, the modified factor VIII polypeptides provided herein offer factor VIII with advantages including a decrease in the frequency of injections needed to maintain a sufficient drug level in serum, thus leading to, for example, higher comfort and acceptance by subjects, lower doses necessary to achieve comparable biological effects and attenuation of secondary effects.

Provided herein are modified factor VIII polypeptides containing modifications that alter any one or more of the properties of factor VIII that contribute to reduced immunogenicity/antigenicity. Reduced immunogenicity/antigenicity can be accomplished by amino acid replacement. Generally, modified factor VIII polypeptides retain one or more activities of an unmodified factor VIII polypeptide. For example, the modified factor VIII polypeptides provided herein exhibit at least one activity that is substantially unchanged (less than 1%, 5% or 10% changed) compared to the unmodified or wild-type factor VIII. In other examples, the activity of a modified factor VIII polypeptide is increased or is decreased as compared to an unmodified factor VIII polypeptide. In another embodiment, the modified factor VIII polypeptides provided herein can inhibit an activity of the unmodified and/or wild-type native factor VIII polypeptide. Activity includes, for example, but not limited to blood coagulation, platelet binding, cofactor binding and protease activity. Activity can be assessed in vitro or in vivo and can be compared to the unmodified factor VIII polypeptide.

Modified factor VIII polypeptides provided herein can be modified at one or more amino acid positions corresponding to amino acid positions of an unmodified factor VIII polypeptide, for example, a factor VIII polypeptide having an amino acid sequence set forth in SEQ ID NO: 1. See Table A. SEQ ID NO:2 is one embodiment of a modified factor VIII polypeptide, where X is any amino acid and at least one X is a modified amino acid. See Table A. Modified factor VIII polypeptides provided herein include human factor VIII (hFactor VIII) variants. A hfactor VIII polypeptide can be of any human tissue or cell-type origin. Modified factor VIII polypeptides provided herein also include variants of factor VIII of non-human origin. Modified factor VIII polypeptides also include polypeptides that are hybrids of different factor VIII polypeptides and also synthetic factor VIII polypeptides prepared recombinantly or synthesized or constructed by other methods known in the art based upon known polypeptides.

TABLE A

| | | SEQ |
|---|---|---|
| | | ID |
| Description | Sequence | NO |
| Factor VIII | MQIELSTCFFLCLLRFCFSATRRYYLGAVELSWDYMQSDLGELP | 1 |
| Polypeptide | VDARFPPRVPKSFPFNTSVVYKKTLFVEFTDHLFNIAKPRPPWMGLLGPTIQAEVYDT | |
| (NM_000132.2) | VVITLKNMASHPVSLHAVGVSYWKASEGAEYDDQTSQREKEDDKVFPGGSHTYVWQVL | |
| | KENGPMASDPLCLTYSYLSHVDLVKDLNSGLIGALLVCREGSLAKEKTQTLHKFILLF | |
| | AVFDEGKSWHSETKNSLMQDRDAASARAWPKMHTVNGYVNRSLPGLIGCHRKSVYWHV | |
| | IGMGTTPEVHSIFLEGHTFLVRNHRQASLEISPITFLTAQTLLMDLGQFLLFCHISSH | |
| | QHDGMEAYVKVDSCPEEPQLRMKNNEEAEDYDDDLTDSEMDVVRFDDDNSPSFIQIRS | |
| | VAKKHPKTWVHYIAAEEEDWDYAPLVLAPDDRSYKSQYLNNGPQRIGRKYKKVRFMAY | |
| | TDETFKTREAIQHESGILGPLLYGEVGDTLLIIFKNQASRPYNIYPHGITDVRPLYSR | |
| | RLPKGVKHLKDFPILPGEIFKYKWTVTVEDGPTKSDPRCLTRYYSSFVNMERDLASGL | |
| | IGPLLICYKESVDQRGNQIMSDKRNVILFSVFDENRSWYLTENIQRFLPNPAGVQLED | |
| | PEFQASNIMHSINGYVFDSLQLSVCLHEVAYWYILSIGAQTDFLSVFFSGYTFKHKMV | |
| | YEDTLTLFPFSGETVFMSMENPGLWILGCHNSDFRNRGMTALLKVSSCDKNTGDYYED | |
| | SYEDISAYLLSKNNAIEPRSFSQNSRHPSTRQKQFNATTIPENDIEKTDPWFAHRTPM | |
| | PKIQNVSSSDLLMLLRQSPTPHGLSLSDLQEAKYETFSDETSPGAIDSNNSLSEMTHF | |
| | RPQLHHSGDMVFTPESGLQLRLNEKLGTTAATELKKLDFKVSSTSNNLISTIPSDNLA | |
| | AGTDNTSSLGPPSMPVHYDSQLDTTLFGKKSSPLTESGGPLSLSEENNDSKLLESGLM | |
| | NSQESSWGKNVSSTESGRLFKGKRAHGPALLTKDNALFKVSISLLKTNKTSNNSATNR | |
| | KTHIDGPSLLIENSPSVWQNILESDTEFKKVTPLIHDRMLMDKNATALRLNHMSNKTT | |
| | SSKNMEMVQQKKEGPIPPDAQNPDMSFFKMLFLPESARWIQRTHGKNSLNSGQGPSPK | |
| | QLVSLGPEKSVEGQNFLSEKNKVVVGKGEFTKDVGLKEMVFPSSRNLFLTNLDNLHEN | |
| | NTHNQEKKIQEEIEKKETLIQENVVLPQIHTVTGTKNFMKNLFLLSTRQNVEGSYDGA | |
| | YAPVLQDFRSLNDSTNRTKKHTAHFSKKGEEENLEGLGNQTKQIVEKYACTTRISPNT | |
| | SQQNFVTQRSKRALKQFRLPLEETELEKRIIVDDTSTQWSKNMKHLTPSTLTQIDYNE | |
| | KEKGAITQSPLSDCLTRSHSIPQANRSPLPIAKVSSFPSIRPIYLTRVLFQDNSSHLP | |
| | AASYRKKDSGVQESSHFLQGAKKNNLSLAILTLEMTGDQREVGSLGTSATNSVTYKKV | |
| | ENTVLPKPDLPKTSGKVELLPKVHIYQKDLFPTETSNGSPGHLDLVEGSLLQGTEGAI | |
| | KWNEANRPGKVPFLRVATESSAKTPSKLLDPLAWDNHYGTQIPKEEWKSQEKSPEKTA | |
| | FKKKDTILSLNACESNHAIAAINEGQNKPEIEVTWAKQGRTERLCSQNPPVLKRHQRE | |
| | ITRTTLQSDQEEIDYDDTISVEMKKEDFDIYDEDENQSPRSFQKKTRHYFIAAVERLW | |
| | DYGMSSSPHVLRNRAQSGSVPQFKKVVFQEFTDGSFTQPLYRGELNEHLGLLGPYIRA | |
| | EVEDNIMVTFRNQASRPYSFYSSLISYEEDQRQGAEPRKNFVKPNETKTYFWKVQHHM | |
| | APTKDEFDCKAWAYFSDVDLEKDVHSGLIGPLLVCHTNTLNPAHGRQVTVQEFALFFT | |
| | IFDETKSWYFTENMERNCRAPCNIQMEDPTFKENYRFHAINGYIMDTLPGLVMAQDQR | |
| | IRWYLLSMGSNENIHSIHFSGHVFTVRKKEEYKMALYNLYPGVFETVEMLPSKAGIWR | |
| | VECLIGEHLHAGMSTLFLVYSNKCQTPLGMASGHIRDFQITASGQYGQWAFKLARLHY | |
| | SGSINAWSTKEPFSWIKVDLLAPMIIHGIKTQGARQKFSSLYISQFIIMYSLDGKKWQ | |
| | TYRGNSTGTLMVFFGNVDSSGIKHNIFNPPIIARYIRLHPTHYSIRSTLRMELMGCDL | |
| | NSCSMPLGMESKAISDAQITASSYFTNMFATWSPSKARLHLQGRSNAWRPQVNNPKEW | |
| | LQVDFQKTMKVTGVTTQGVKSLLTSMYVKEFLISSSQDGHQWTLFFQNGKVKVFQGNQ | |
| | DSFTPVVNSLDPPLLTRYLRIHPQSWVHQIALRMEVLGCEAQDLY | |
| Modified | MQIELSTCFFLCLLRFCFSATRRYYLGAVELSWDYMQSDLGELP | 2 |
| Factor VIII | VDARFPPRVPKSFPFNTSVVYKKTLFVEFTDHLFNIAKPRPPWMGLLGPTIQAEVYDT | |
| Polypeptide; | VVITLKNMASHPVSLHAVGVSYWKASEGAEYDDQTSQREKEDDKVFPGGSHTYVWQVL | |
| X is any | KENGPMASDPLCLTYSYLSHVDLVKDLNSGLIGALLVCREGSLAKEKTQTLHKFILLF | |
| amino acid | AVFDEGKSWHSETKNSLMQDRDAASARAWPKMHTVNGYVNRSLPGLIGCHRKSVYWHV | |
| and at least | IGMGTTPEVHSIFLEGHTFLVRNHRQASLEISPITFLTAQTLLMDLGQFLLFCHISSH | |
| one X is a | QHDGMEAYVKVDSCPEEPQLRMKNNEEAEDYDDDLTDSEMDVVRFDDDNSPSFIQIRS | |
| modified | VAKKHPKTWVHYIAAEEEDWDYAPLVLAPDDRSYKSQYLNNGPQRIGRKYKKVRFMAY | |
| amino acid | TDETFKTREAIQHESGILGPLLYGEVGDTLLIIFKNQASRPYNIYPHGITDVRPLYSR | |
| | RLPKGVKHLKDFPILPGEIFKYKWTVTVEDGPTKSDPRCLTRYYSSFVNMERDLASGL | |
| | IGPLLICYKESVDQRGNQIMSDKRNVILFSVFDENRSWYLTXXXXXXXXXXXXXXXXX | |
| | XXFQASNIMHSINGYVFDSLQLSVCLHEVAYWYILSIGAQTDFLSVFFSGYTFKHKMV | |
| | YEDTLTLFPFSGETVFMSMENPGLWILGCHNSDFRNRGMTALLKVSSCDKNTGDYYED | |
| | SYEDISAYLLSKNNAIEPRSFSQNSRHPSTRQKQFNATTIPENDIEKTDPWFAHRTPM | |
| | PKIQNVSSSDLLMLLRQSPTPHGLSLSDLQEAKYETFSDDPSPGAIDSNNSLSEMTHF | |
| | RPQLHHSGDMVFTPESGLQLRLNEKLGTTAATELKKLDFKVSSTSNNLISTIPSDNLA | |
| | AGTDNTSSLGPPSMPVHYDSQLDTTLFGKKSSPLTESGGPLSLSEENNDSKLLESGLM | |
| | NSQESSWGKNVSSTESGRLFKGKRAHGPALLTKDNALEKVSISLLKTNKTSNNSATNR | |
| | KTHIDGPSLLIENSPSVWQNILESDTEFKKVTPLIHDRMLNDKNATALRLNHMSNKTT | |
| | SSKNMEMVQQKKEGPIPPDAQNPDMSFFKMLFLPESARWIQRTHGKNSLNSGQGPSPK | |
| | QLVSLGPEKSVEGQNFLSEKNKVVVGKGEFTKDVGLKEMVFPSSRNLFLTNLDNLHEN | |
| | NTHNQEKKIQEEIEKKETLIQENVVLPQIHTVTGTKNFMKNLFLLSTRQNVEGSYDGA | |
| | YAPVLQDFRSLNDSTNRTKKHTAHFSKKGEEENLEGLGNQTKQIVEKYACTTRISPNT | |
| | SQQNFVTQRSKRALKQFRLPLEETELEKRIIVDDTSTQWSKNMKHLTPSTLTQIDYNE | |
| | KEKGAITQSPLSDCLTRSHSIPQANRSPLPIAKVSSFPSIRPTYLTRVLFQDNSSHLP | |
| | AASYRKKDSGVQESSHFLQGAKKNNLSLAILTLEMTGDQREVGSLGTSATNSVTYKKV | |
| | ENTVLPKPDLPKTSGKVELLPKVHIYQKDLFPTETSNGSPGHLDLVEGSLLQGTEGAI | |
| | KWNEANRPGKVPFLRVATESSAKTPSKLLDPLAWDNHYGTQIPKEEWKSQEKSPEKTA | |
| | FKKKDTILSLNACESNHAIAAINEGQNKPEIEVTWAKQGRTERLCSQNPPVLKRHQRE | |
| | ITRTTLQSDQEEIDYDDTISVEMKKEDFDIYDEDENQSPRSFQKKTRHYFIAAVERLW | |
| | DYGMSSSPHVLRNRAQSGSVPQFKKVVFQEFTDGSFTQPLYRGELNEHLGLLGPYIRA | |
| | EVEDNIMVTFRNQASRPYSFYSSLISYEEDQRQGAEPRKNFVKPNETKTYFWKVQHHM | |
| | APTKDEFDCKAWAYFSDVDLEKDVHSGLIGPLLVCHTNTLNPAHGRQVTVQEFALFFT | |
| | IFDETKSWYFTENMERNCRAPCNIQMEDPTFKENYRFHAINGYIMDTLPGLVMAQDQR | |
| | IRWYLLSMGSNENIHSIHFSGHVFTVRKKEEYKMALYNLYPGVFETVEMLPSKAGIWR | |
| | VECLIGEHLHAGMSTLFLVYSNKCQTPLGMASGHIRDFQITASGQYGQWAPKLARLHY | |
| | SGSINAWSTKEPFSWIKVDLLAPMIIHGIKTQGARQKFSSLYISQFIIMYSLDGKKWQ | |
| | TYRGNSTGTLMVFFGNVDSSGIKHNIFNPPIIARYIRLHPTHYSIRSTLRMELMGCDL | |
| | NSCSMPLGMESKAISDAQITASXXXXXXXXXXXXXXXXXXXXXXXXXXXXQVNNPKEW | |
| | LQVDFQKTMKVTGVTTQGVKSLLTSMYVKEFLISSSQDGHQWTLFFQNGKVKVFQGNQ | |
| | DSFTPVVNSLDPPLLTRYLRIHPQSWVHQIALRMEVLGCEAQDLY | |

Also among the variants provided herein are modified factor VIII polypeptides with two or more modifications compared to native or wild-type factor VIII. Modified factor VIII polypeptides include those with 2, 3, 4, 5, 6, 7, 8, 9, 10, 11, 12, 13, 14, 15, 16, 17, 18, 19, 20 or more modified positions.

Typically, modifications include replacement (substitution), addition, deletion or a combination thereof, of amino acid residues as described herein. Generally, the modification results in reduced immunogenicity/antigenicity without losing at least one activity, of an unmodified factor VIII polypeptide. Exemplary epitopes for amino acid modification corresponding to amino acid positions of a mature factor VIII polypeptide (e.g., SEQ ID NO:1) that can contribute to reducing immunogenicity/antigenicity are set forth in Table B.

TABLE B

| Epi- | | FVIII | | |
|---|---|---|---|---|
| tope | Type | Domain | FVIII Residues | Minimal |
| 1 | T-cell | C2(2173- | 2194-2213 | S2194-P2205 |
| | (A2201P mild | 2332) | (SYFTNMFATWSP | (SYFTNMFA |
| | hemophilia) | | SKARLHLQ)(SEQ | TWSP)(SEQ |
| | | | ID NO: 3) | ID NO: 4) |
| 2 | T-cell | C2(2173- | 2202-2221 | |
| | (A2201P mild | 2332) | (TWSPSKARLHLQ | |
| | hemophilia) | | GRSNAWRP)(SEQ | |
| | | | ID NO: 5) | |
| 3 | T-cell | A2(373- | 589-608 | 594-602 |
| | (R593C mild | 740) | (ENIQRFLPNPAG | (FLPNPAGV |
| | hemophilia) | | VQLEDPE)(SEQ | Q)(SEQ ID |
| | | | ID NO: 6) | NO: 7) |
| 4 | B-cell | C2(2173- | | |
| | (IgG4 | 2332) | | |
| | antibody | | | |
| | BO2C11) | | | |
| 5 | B-cell | C2(2173- | | |
| | | 2332) | | |

A modified factor VIII polypeptide exhibiting a modified P-22DN immunogenicity/antigenicity may be produced by changing an identified epitope area of an unmodified factor VIII polypeptide by, e.g., genetically engineering a mutation in a epitope sequence encoding the unmodified factor VIII polypeptide.

An epitope in a factor VIII polypeptide may be changed by substituting at least one amino acid of the epitope area. In an embodiment at least one amino acid deemed important for HLA-class T1 receptor (e.g., DR) contact is modified. In an embodiment at least one amino acid deemed important for TCR contact is modified. In an embodiment at least one amino acid deemed important for antibody contact is modified. In an embodiment at least one amino acid deemed important for class II or TCR contact is modified and at least one amino acid deemed important for antibody contact is modified. The change will often be substituting to an amino acid of different size, hydrophilicity, and/or polarity, such as a small amino acid versus a large amino acid, a hydrophilic amino acid versus a hydrophobic amino acid, a polar amino acid versus a non-polar amino acid and a basic versus an acidic amino acid.

Other changes may be the addition/insertion or deletion of at least one amino acid of the epitope sequence, e.g., deleting an amino acid important for class II or TCR recognition and activation and/or antibody binding. Furthermore, an epitope area may be changed by substituting some amino acids, and deleting/adding one ore more others.

When one uses protein engineering to alter or eliminate epitopes, it is possible that new epitopes are created, or existing epitopes are duplicated. To reduce this risk, one can map the planned mutations at a given position on the 3-dimensional structure of the protein of interest, and control the emerging amino acid constellation against a database of known epitope patterns, to rule out those possible replacement amino acids, which are predicted to result in creation or duplication of epitopes. Thus, risk mutations can be identified and eliminated by this procedure, thereby reducing the risk of making mutations that lead to increased rather than decreased immunogenicity/antigenicity.

A modified factor VIII polypeptide exhibiting a modified immunogenicity/antigenicity may be produced by chemically modifying (e.g., via conjugation) the identified epitope area of the unmodified factor VIII polypeptide. For example, the factor VIII polypeptide can be incubated with an active or activated polymer and subsequently separated from the unreacted polymer. This can be done in solution followed by purification or it can conveniently be done using the immobilized protein variants, which can easily be exposed to different reaction environments and washes.

Thus, modified factor VIII polypeptides of the invention can be modified within one or more epitopes described herein via, e.g., amino acid additions, substitutions, or deletions. In addition, modification can include chemical conjugation to one or more epitopes described herein. In some embodiments, a modification is made in a T cell epitope. In some embodiments, a modification is made in a B cell epitope. In some embodiments, a modification is made in both a T cell epitope and a B cell epitope.

Methods of Making Factor VIII Polypeptides

The factor VIII polypeptides of this invention largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host and/or cell. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host and/or cell. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149: Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Pharmaceutical Compositions and Therapeutic Methods of Use

In some embodiments, a modified factor VIII polypeptide is administered to a subject in need thereof to reduce or prevent a condition associated with an immune response to factor VIII. In some embodiments, a modified factor VIII polypeptide is administered to a subject in need thereof to treat or reduce a condition associated with an immune response to factor VIII.

In certain embodiments, a factor VIII polypeptide is administered alone. In certain embodiments, a factor VIII polypeptide is administered prior to the administration of at least one other therapeutic agent. In certain embodiments, a factor VIII polypeptide is administered concurrent with the administration of at least one other therapeutic agent. In certain embodiments, a factor VIII polypeptide is administered subsequent to the administration of at least one other therapeutic agent. In other embodiments, a factor VIII polypeptide is administered prior to the administration of at least one other therapeutic agent. As will be appreciated by one of skill in the art, in some embodiments, the factor VIII polypeptide is combined with the other agent/compound. In some embodiments, the factor VIII polypeptide and other agent are administered concurrently. In some embodiments, the factor VIII polypeptide and other agent are not administered simultaneously; with the factor VIII polypeptide being administered before or after the agent is administered. In some embodiments, the subject receives both the factor VIII polypeptide and the other agent during a same period of prevention, occurrence of a disorder, and/or period of treatment.

Pharmaceutical compositions of the invention can be administered in combination therapy. i.e., combined with other agents. In certain embodiments, the combination therapy comprises nuclease molecule, in combination with at least one other agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the invention provides for pharmaceutical compositions comprising a factor VIII polypeptide together with a pharmaceutically acceptable diluent, carrier, solubilizer, emulsifier, preservative and/or adjuvant.

In certain embodiments, the invention provides for pharmaceutical compositions comprising a factor VIII polypeptide and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides: and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, a factor VIII polypeptide and/or a therapeutic molecule is linked to a half-life extending vehicle known in the art. Such vehicles include, but are not limited to, polyethylene glycol, glycogen (e.g., glycosylation of the factor VIII polypeptide), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a factor VIII polypeptide, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a factor VIII polypeptide, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired factor VIII polypeptide, with or without additional therapeutic agents, in a pharmaceutically acceptable vehicle. In certain embodiments, a vehicle for parenteral injection is sterile distilled water in which a factor VIII polypeptide, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a factor VIII polypeptide, with or without at least one additional therapeutic agent, can be formulated as a dry powder for inhalation. In certain embodiments, an inhalation solution comprising a factor VIII polypeptide, with or without at least one additional therapeutic agent, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, a factor VIII polypeptide, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of a factor VIII polypeptide and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a factor VIII polypeptide, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving a factor VIII polypeptide, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133.988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising a factor VIII polypeptide, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which a factor VIII polypeptide, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of a factor VIII polypeptide and/or any additional therapeutic agents in the formulation used. In certain embodiments, a clinician will administer the composition until a dosage is reached that achieves the desired effect. In certain embodiments, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a factor VIII polypeptide, with or without at least one additional therapeutic agent, in an ex vivo manner. In such instances, cells, tissues and/or organs that have been removed from the patient are exposed to a pharmaceutical composition comprising a factor VIII polypeptide, with or without at least one additional therapeutic agent, after which the cells, tissues and/or organs are subsequently implanted back into the patient.

In certain embodiments, a factor VIII polypeptide and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semipermeable polymeric enclosures or membranes that allow the release of the protein product(s) but prevent the destruction of the cells by the patient's immune system or by other detrimental factors from the surrounding tissues.

The modified factor VIII polypeptides and nucleic acid molecules provided herein can be used for treatment of any condition for which unmodified factor VIII is employed. Modified factor VIII polypeptides have therapeutic activity alone or in combination with other agents. The modified factor VIII polypeptides provided herein are designed to retain therapeutic activity but exhibit modified properties, particularly reduced immunogenicity/antigenicity. Such modified properties, for example, can improve the therapeutic effectiveness of the polypeptides and/or can provide for additional routes of administration.

In particular, modified factor VIII polypeptides, are intended for use in therapeutic methods in which factor VIII has been used for treatment. Such methods include, but are not limited to, methods of treatment of diseases and disorders, such as, but not limited to, hemophilias. Modified factor VIII polypeptides also can be used in the treatment of additional bleeding diseases and disorders where deemed efficacious by one of skill in the art.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Materials and Methods

Antibodies.

Ten murine mAbs were selected from 56 mAbs characterized earlier using ELISA assays¹³ as representative of Type A, AB, B, BC and C inhibitors. Murine anti-FVIII C2 domain mAbs ESH4 and ESH8 were from American Diagnostica (Stamford, Conn.), while mAbs 3E6 (GMA-8013), I54, I109, 1B5 (GMA-8008), 3D12, 3G6 (GMA-8014), 2-77 (GMA-8006) and 2-117 (GMA-8003) were prepared as described previously¹³ or were kindly provided by Dr. William Church (Green Mountain Antibodies, Burlington, Vt.). The human anti-FVIII mAb BO2C11 was kindly provided by Dr. Marc Jacquemin. Goat anti-mouse IgG, Fc-γ (115-005-071) was from Jackson ImmunoResearch (Westgrove, Pa.).

FVIII-C2 Proteins and SPR Measurements.

T FVIII-C2 proteins were expressed in E. coli purified and analyzed by SPR as described in greater detail below. Briefly, SPR measurements were carried out on a Biacore T100 instrument (GE Healthcare Life Sciences) under standard conditions (25° C. and 1 atm). Goat anti-mouse IgG specifically directed towards the Fc-gamma fragment was immobilized covalently on all channels of a CM5 chip by amine derivatization. Murine anti-FVIII mAb stock solutions were injected over the sensor in three flow channels, while the fourth channel served as a reference. BO2C11-Fab was immobilized covalently by amine derivatization. Single-cycle kinetics experiments¹⁵ were carried out in which wild-type or mutant FVIII-C2 proteins were injected serially over the biosensor surfaces at increasing concentrations, without regenerating the biosensor surface after each injection, followed by a 30-60 minute buffer injection to measure dissociation rates. The association (kₐ) and dissociation (k_{d}) rate constants for binding of WT-FVIII-C2 were measured during each set of SPR runs and the resulting k_{d} values used to compute the k_{d}(mutein)/k_{d}(WT) ratios for that set of muteins. FVIII-C2 muteins with a k_{d}>2.0× the k_{d} for WT-FVIII-C2 were considered candidates for B-cell epitope residues. For each of the mAbs, the rate constants for the binding of WT-FVIII-C2 were determined by averaging the results obtained from at least three SPR runs. Dissociation rate constants (k_{d}), rather than affinities, were chosen as the most relevant metric for identifying “functional B-cell epitopes” because the residence time (1/k_{d} for a bimolecular interaction) of an antibody-antigen complex indicates its maximum potential lifetime in the circulation. Analysis of residence times is widely utilized in lead optimization studies of potential inhibitory drug targets¹⁶⁻¹⁹.

Reagents:

BugBuster Extraction reagent, E. coli strain OrigamiB(DE3)pLysS, expression vector pET-16b(+), Benzonase Nuclease and rLysozyme Solution were from Novagen, Inc. (San Diego, Calif.), and buffers used for purification were made according to the instructions in the BugBuster kit. Nickel sulfate, TRIZMA base, NaCl and EDTA were from Sigma-Aldrich (St. Louis, Mo.). Luria Broth (LB) and Super Broth were from Becton Dickinson and Company (Franklin Lakes, N.J.). SOC medium and carbenicillin were from Mediatech, Inc. (Herndon, Va.), chloramphenicol from Sigma-Aldrich, 10×DPBS and isopropylthio-β-galactoside (IPTG) from Life Technologies (Grand Island, N.Y.). Restriction enzymes NdeI and BamHI were from New England Biolabs (Ipswich, Mass.). PCR primers were from Invitrogen (Grand Island, N.Y.), Midland Certified (Midland, Tex.), or synthesized in-house. Miniprep kits were from Qiagen (Valencia, Calif.). QuikChange kits and XL10-Gold ultracompetent cells were from Stratagene (Agilent Technologies, Santa Clara, Calif.). Ni-NTA His-Bind purification columns were from EMD Chemicals (Gibbstown, N.J.). N-lauroylsarcosine sodium salt was from TCI America, Triton X-114 from Sigma-Aldrich, and glycerol from MP BioMedicals (Solon, Ohio). Immobilization reagents 1-ethyl-3-(3-dimethylaminopropyl) carbodiimide HCl (EDC), N-hydroxysuccinimide (NHS), ethanolamine, CM5 sensor chips. HBS-EP+ running buffer (10 mM HEPES, 150 mM NaCl, 3 mM EDTA, 0.05% (v/v) surfactant P20, pH 7.4) and 10 mM sodium acetate pH 5 were from GE Healthcare Life Sciences (Piscataway, N.J.).

FVIII-C2 Proteins:

FVIII-C2 proteins were produced in E. coli as follows: The wild-type C2 (WT-FVIII-C2) sequence, consisting of residues 2170-2332, was amplified from a puc18-C2 plasmid using PCR primers introducing a 5′ NdeI restriction site and a 3′ BamHI restriction site: forward: 5′-GGCGCGCATATGGATTTAAATAGTTGCAGCATG; reverse: 3′-GGCGCGGGATCCCTAGTAGAGGTCCTGTGC. The PCR product was digested with NdeI and BamHI and subcloned into expression vector pET-16b(+), then linearized by digestion with the same enzymes to make pET16b-WTC2, which has an N-terminal extension of 10 His residues. Mutations to introduce single amino acid substitutions were designed after calculating the solvent exposures of all amino acid residues from the FVIII-C2 domain crystal structure using the program Stride. Sixty-six sites with surface-exposed side chains, distributed over all faces of the protein, were selected for mutagenesis. Several polar or charged residues that were mostly buried (e.g. E2228 and S2254) were included due to their ability to form hydrogen or ionic bonds at the protein surface. The corresponding pET-16b-C2 plasmid constructs were generated using the QuikChange PCR protocol and transformed into XL10-Gold ultracompetent cells. Mutagenesis primers are listed in Table 3. The plasmids were purified by minipreps and all FVIII-C2 sequences verified by DNA sequencing. The expression strain E. coli OrigamiB(DE3)pLysS was transfected by adding 20 μl of a log phase (A₆₀₀˜0.6) culture grown in LB to 1 μl of each pET-16b-C2 plasmid (miniprep DNA diluted 1:5 in water), incubating this mixture for 30 s at 42° C. followed by 2 min on ice. 80 μl SOC medium was added and cultures shaken at 37° C. for 1 hr and plated on LB/agar plates containing 75 μg/mL carbenicillin, 34 μg/mL chloramphenicol. The plates were incubated at 37° C. overnight, then five colonies were picked for each mutant and 10-mL cultures were grown overnight in LB plus carbenicillin (75 μg/mL) and chloramphenicol (34 μg/mL). Three mL of each culture was added to 150 mL Super Broth and shaken at 37° C. to log-phase growth, 45 μl 1M IPTG was added, and the culture was shaken for 15-20 min at 37° C., then at 30° C. overnight. Cells were pelleted at 4000×g for 20 min, the supernatant discarded, and the pellets transferred to 50 mL conical tubes, which were weighed and then placed in liquid nitrogen for 20 min. For each gram of pellet, 4.7 mL Bug Buster extraction reagent containing 1250 U benzonase, 48 kU rLysozyme and 2.7% glycerol was used. Cells were carefully re-suspended by stirring for 20-60 min at room temperature and then centrifuged at 16,000×g for 30 min at 4° C. The supernatant was passed through a 0.2 μm filter and applied to a His-Bind purification column. Purification and dialysis were performed at 4° C. The column was charged and equilibrated by washing with water (5 column volumes (CV)), charge buffer (50 mM NiSO₄, 2.5% glycerol, 5 CV) and binding buffer (20 mM Tris-HCl, 0.5 M NaCl, 5 mM imidazole, 2.5% glycerol, 0.3% N-lauroylsarcosine, pH 7.9, 5 CV). The cell lysate was next loaded on the column and the column was washed with binding buffer (10 CV), wash buffer (20 mM Tris-HCl, 0.5 M NaCl, 60 mM imidazole, pH 7.9) containing 0.1% Triton X-114 (20 CV), wash buffer containing 2.5% glycerol (20 CV) and elution buffer (20 mM Tris-HCl, 0.5 M NaCl, 1 M imidazole, 2.5% glycerol, pH 7.9, 10 CV). The eluate was dialyzed against 3 L of cold, sterile 1×DPBS (137.9 mM NaCl, 2.7 mM KCl, 8.1 mM Na₂HPO₄, 1.5 mM KH₂PO₄, pH 7.1) containing 5% glycerol three times and sterile filtered (0.2 μm). Absorbance of FVIII-C2 protein solutions at 280 nm was determined using a NanoDrop 1000 spectrophotometer (Thermo Fisher, Wilmington, Del.) and concentrations were estimated using a calculated extinction coefficient of ε^{280nm,0.1%}=1.8. All proteins were stored as aliquots at −80° C. until analysis. Prior to SPR measurements, proteins were run again over Ni-NTA His-Bind resin, as this step was found to remove minor contaminants causing nonspecific binding to the sensor surface, and their concentrations were then re-determined as described above.

Surface Plasmon Resonance:

SPR measurements were carried out on a Biacore T100 instrument (GE Healthcare Life Sciences) at 25° C. Goat anti-mouse IgG, Fc-γ fragment was immobilized covalently on a CM5 chip by amine derivatization, following the manufacturer's suggested protocol. Briefly, 0.5 M EDC and 0.1 M NHS were injected to activate the CM5 biosensor surface. The anti-mouse IgG, Fc-γ was diluted to 5 μg/mL in 10 mM sodium acetate pH 5.0 and then injected serially in HBS-EP+buffer until approximately 9000 RUs were recorded, at which point 35 μl ethanolamine was injected to quench the reactive sites on the surface. All FVIII-C2 protein and antibody dilutions were prepared using running buffer HBS-EP+. Murine anti-FVIII mAb stock solutions were diluted to 10 μg/mL in HBS-EP+ and injected over the sensor surface. Three different mAbs were captured in this manner in three flow channels, while the goat anti-mouse IgG. Fc-γ fragment immobilized in the fourth channel served as a reference. The dissociation rates for the mAbs captured on the Fc-γ fragment were determined (not shown) and found to be slow compared to those of FVIII-C2 proteins bound to the captured antibodies. The drift of blank runs was reproducible over the course of the assay, ranging between 1-2 RUs. Baseline drift was corrected by subtracting the signals from blank runs in the reference channel. Wild-type and mutant FVIII-C2 proteins were introduced as sequential injections (for single cycle kinetics analysis) over all four channels at concentrations 0.9, 1.9, 3.8, 7.5 and 15 nM, resulting in capture of 60-180 RU of the FVIII-C2 proteins at the highest (15 nM) concentration. HBS-EP+ running buffer was then injected for 30 min to allow adequate time to measure the dissociation rate constants (k_{d}) accurately. Regeneration of the goat anti-mouse IgG. Fc-γ surface was achieved with six 20 s injections of 20 mM HCl. The flow rate was 50 UL/min throughout the analysis. Resonance signals (RU) after regeneration injections were monitored to ensure complete dissociation of the FVIII-C2 proteins before initiating the next experiment. Injections of HBS-EP+ running buffer were also performed periodically in order to subtract instrument noise and drift. The level of nonspecific binding to the IgG, Fc-γ reference channel was confirmed to be minimal. Binding curves were processed by subtracting the signals from the reference channel and the buffer injections. The Fab fragment of human mAb BO2C11 was immobilized covalently following the manufacturer's suggested protocol, and a similar series of injections of FVIII-C2 proteins was performed and analyzed.

Data analysis was performed using the Biacore T100 Evaluation software version 2.0.3. Based on the good agreement between the theoretical Rmax determined by the evaluation software and the measured Rmax values, a 1:1 binding model was determined to be appropriate for these experiments. The binding of WT-FVIII-C2 was measured during each set of SPR runs and the resulting k_{d} value used to compute the k_{d}(mutein)/k_{d}(WT) ratios for that set of muteins. FVIII-C2 muteins with a dissociation rate constant (k_{d})>2.0× the k_{d} for WT-FVIII-C2 were considered candidates for B-cell epitope residues. For each of the mAbs, the average association (kₐ) and dissociation (k_{d}) rate constants for the binding of WT-FVIII-C2 were determined by averaging the results obtained from at least three SPR runs. The resulting kinetic constants and standard deviations (Table 2) indicate the reproducibility of the measurements.

Visualization of B-Cell Epitopes.

After the SPR data were collected the crystal structures of FVIII-C²⁰ and B-domain-deleted FVIII^{21,22} were visualized using the graphics program PyMOL²³ to localize the sites showing altered binding kinetics to the mAbs analyzed herein. The cutoff k_{d} value was chosen empirically, to minimize the number of potential epitope residues located distal from the primary clusters of candidate residues for this series of mAbs.

Results

FVIII-C2 Proteins:

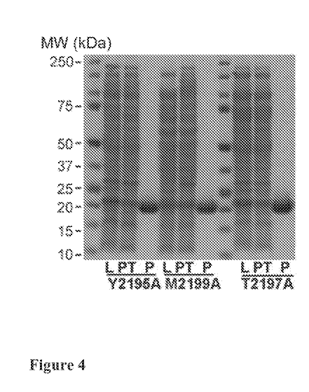

WT-FVIII-C2 and 60 FVIII-C2 muteins were purified to >95% homogeneity (FIG. 4). Six additional FVIII-C2 muteins (S2193A, K2227A, V2232A, K2236A, K2279A and K2281A) were not expressed in a soluble form and were therefore not analyzed. Dynamic light scattering analyses of the purified proteins, carried out for aliquots of each protein preparation both before and after freezing at −80° C., showed a single peak at the expected size for monomeric FVIII-C2 (not shown). Some of the mutant protein preparations showed evidence of higher molecular weight aggregates; these were not analyzed further and were discarded. Multiple aliquots of each well-behaved FVIII-C2 protein preparation were stored frozen to avoid multiple freeze-thaw cycles that could endanger protein structural integrity.

Altered Binding Kinetics Due to Amino Acid Substitutions in FVIII-C2:

The amino acid substitutions affected k_{d} values (relative to the values for WT-FVIII-C2) more than kₐ values in almost all cases. Therefore, a cutoff value based on the ratio of measured k_{d} values of the mutant versus WT protein was chosen to indicate whether the wild-type residues at these positions should be considered as potential contributors to functional B-cell epitopes recognized by the corresponding antibodies. Setting this cutoff value at k_{d}>2.0× the measured k_{d} for WT-FVIII-C2 resulted in the identification of 5-18 residues as candidates for the B-cell epitopes recognized by the 10 murine and one human (BO2C11) mAbs²⁴. Because all of the mAbs were attached on the biosensor surface, the antigen-antibody binding interactions could be modeled as 1:1 interactions (with each Fab region available to bind a single FVIII-C2 protein).

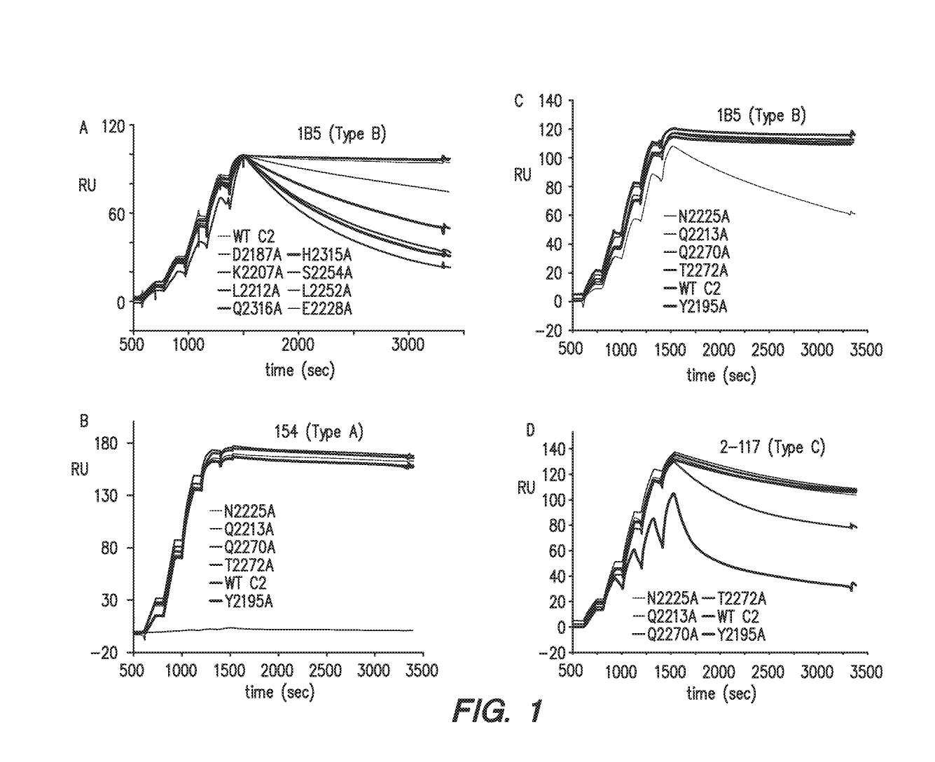

Representative sensorgrams are shown in FIG. 1. Almost all of the FVIII-C2 muteins showed binding kinetics to some or all of the mAbs that were highly similar to the binding of WT-FVIII-C2 (see summary of results in Table 1 and kinetic constants in Table 2), indicating the substitutions did not cause global protein misfolding. Thirty-eight of the 60 muteins tested showed altered binding kinetics restricted to a subset of the mAbs. FVIII-C2-F2200A showed altered kinetics in binding to type AB and type B mAbs (1109, BO2C11, 1B5 and 3D12) with a k_{d}>2.0× that of WT-FVIII-C2. FVIII-C2-R2220A showed altered binding to all mAbs except type A antibodies 3E6 and 154 and type BC antibody 3G6. The ratio [k_{d}(mutein)/k_{d}(WT)] for binding to the third type A mAb, ESH4, was 2.7, and with a more stringent cutoff value for this ratio R2220 would not be identified as part of the epitope recognized by this antibody. Removal of the mostly-buried R2220 side chain would be expected to destabilize the membrane-binding region of FVIII-C2. Therefore its effect on binding kinetics to ESH4 was judged to be a conformational effect and it was not assigned to the epitope for this mAb. The kinetic data from analyzing several preparations of FVIII-C2-F2200A and FVIII-C2-R2220A were more variable and of poorer quality than that of WT-FVIII-C2 and the other muteins, indicating these substitutions altered the protein stability. Nevertheless, the fact that neighboring residues were also pinpointed as parts of epitopes recognized by type AB and B antibodies supported their assignment to these epitopes. Qualitatively, several other FVIII-C2 muteins showed increased antibody-antigen dissociation rates relative to WT-FVIII-C2 although accurate kinetic constants could not be obtained. These results are indicated in Table 1 as “QFD” for “Qualitative Fast Dissociation”.

Identification of B-Cell Epitopes:

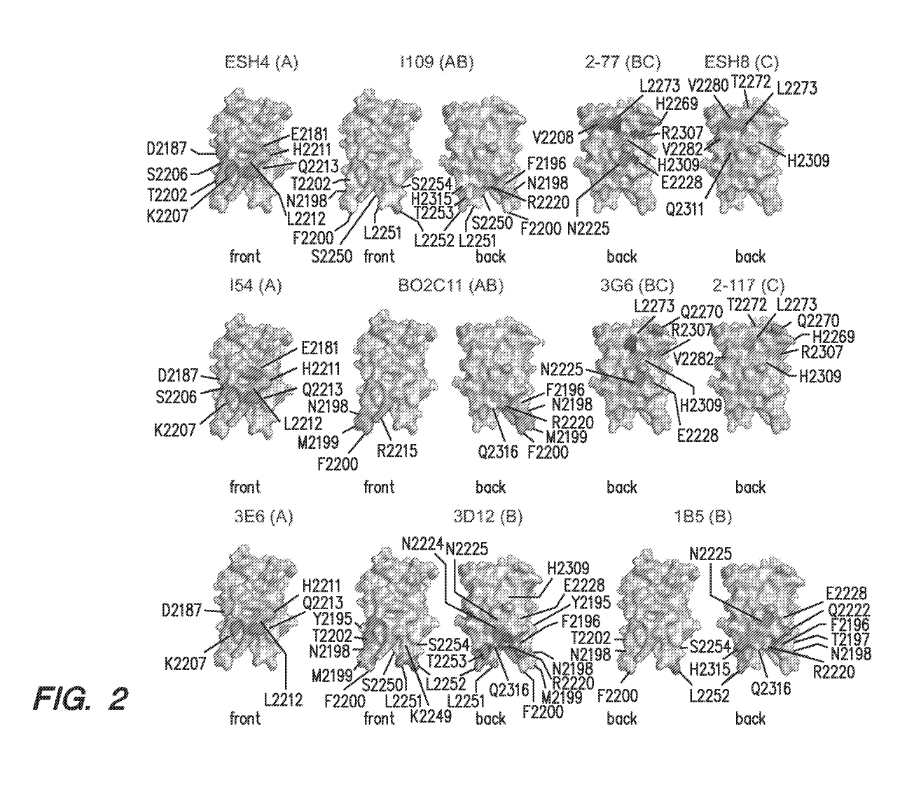

The residues identified by the cutoff criterion of k_{d}(mutein)>2.0×k_{d}(WT), when visualized using PyMOL²³, formed distinct clusters indicating the FVIII-C2 surfaces recognized by each mAb (FIG. 2). Once a cluster was localized to a specific surface region, the full set of FVIII-C2 muteins was not analyzed by SPR as it was considered sufficient to concentrate on residues adjacent to the initial surface thus identified. Substitutions causing altered kinetics at positions that were conformationally non-contiguous with the SPR-identified clusters of potential B-cell epitope residues are noted below as “outliers”. Specific characteristics of the binding interactions in each antibody-antigen complex are described below, with the inhibitory mAbs identified as Type A, AB, B, BC or C according to the criteria of Meeks et al.¹³

Type A Inhibitors:

Three type A inhibitors (ESH4, 3E6 and I54) were evaluated. SPR assays identified the following residues as possibly interacting with all three of these mAbs: D2187, K2207, H2211, L2212 and Q2213. These three epitopes identified kinetically were similar but not identical. Experiments with ESH4 also identified residues E2181, T2202, S2206 and R2220 as possibly contributing to this epitope, but R2220 was excluded subsequently as an outlier, as described above. Experiments with 154 also identified residues E2181 and S2206. These Type A epitopes are immediately adjacent to the beta turn at FVIII residues 2198-2201, which is one of the two “greasy feet” hydrophobic regions of FVIII that bind to phospholipid membranes^{20,25}.

Type AB Inhibitors:

Two type AB inhibitors (I109 and BO2C11) were evaluated. SPR assays identified one cluster of residues as possibly interacting with both of these mAbs: F2196, N2198, F2200 and R2220, I109 also possibly interacted with T2202, S2250, L2251, L2252, T2253, S2254 and H2315, and BO2C11 also possibly interacted with E2181 (outlier), M2199, R2215, Q2270 (outlier) and Q2316. Thus the type AB inhibitors were seen to bind either or both of these hydrophobic beta turns.

Type B Inhibitors:

Two type B inhibitors (1B5 and 3D12) were evaluated. SPR assays identified the following residues as possibly interacting with both of them: F2196, N2198, F2200, T2202, R2220, N2225, E2228, L2252, S2254 and Q2316. Ala substitutions at residues T2197, Q2222, K2239 (outlier) and H2315 also affected binding to 1B5, while Ala substitutions at residues Y2195, M2199, N2224, K2249, S2250, L2251, T2253 and H2309 affected binding to 3D12. The type B epitopes include the two hydrophobic beta turns, as well as migrating further up the “back face” of the molecule to include the loop from N2224-E2228 and H2309. Being of type AB, the epitope of BO2C11 overlaps with the type B inhibitors, 1B5 and 3D12, at one of the hydrophobic beta turns.

Type BC Inhibitors:

Two type BC inhibitors (3G6 and 2-77) were evaluated. SPR indicated the following residues as possibly interacting with both of them: N2225, E2228, L2273, R2307 and H2309. Ala substitutions at residues T2197 (outlier) and Q2270 affected binding only to 3G6, while Ala substitutions at R2220 (outlier), K2239 (outlier). H2269 and V2280 affected binding only to 2-77.

Type C Inhibitors:

Two type C inhibitors (2-117 and ESH8) were evaluated. SPR indicated the following residues as possibly interacting with both 2-117 and ESH8: R2220 (outlier), T2272, L2273, V2282 and H2309. Ala substitutions at residues H2269 and Q2270 affected binding only to 2-117 while Ala substitutions at residues V2280 and Q2311 affected only ESH8 binding. The conservative substitution R2307Q affected binding to mAb 2-117 but not to ESH8.

Modeling of Epitopes in the FVIII Structure:

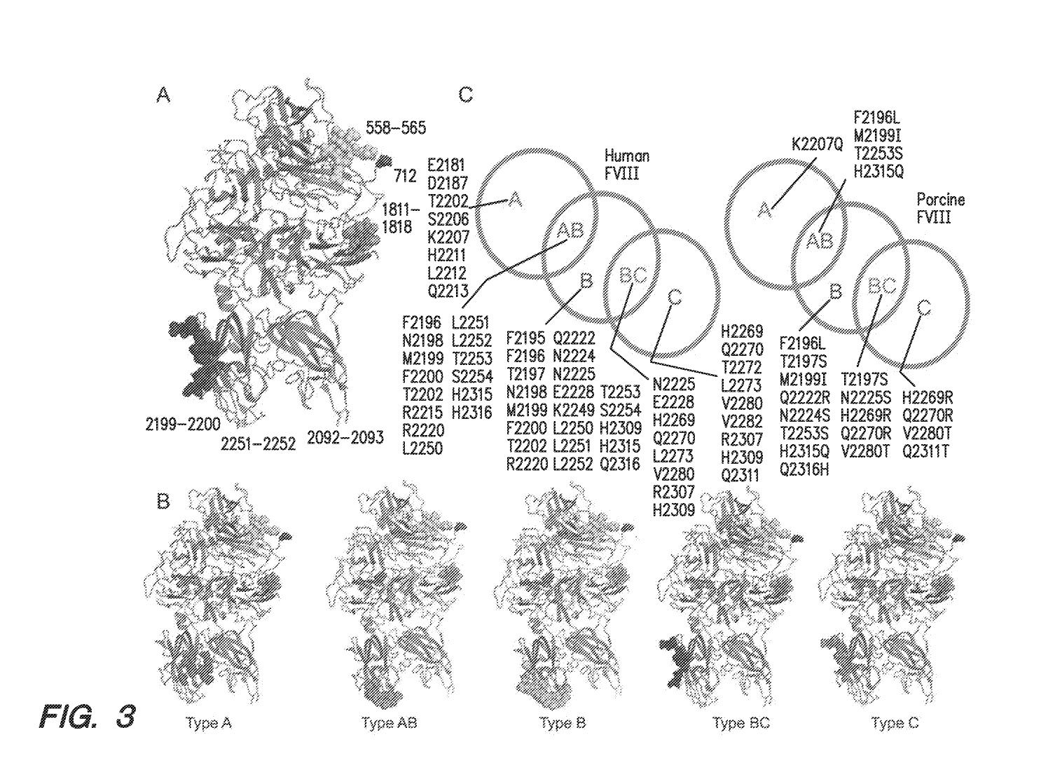

FIG. 3A shows the BC and C epitope residues that comprise “non-classical” inhibitor antibodies, e.g. antibodies that prevent FVIII activation by thrombin and/or FXa. FIG. 3B shows the location of the epitopes recognized by Type A, AB, B, BC and C antibodies.

### DISCUSSION

The SPR experiments identified three distinct clusters of surface-exposed side chains on FVIII-C2 that contributed significant binding avidity for Type A, B and C FVIII-neutralizing antibodies, plus two clusters containing residues that comprised overlap regions for mAb types AB and BC, respectively. SPR experiments were carried out for three type A and two each of types AB, B, BC and C mAbs. The resulting assignments of epitope residues were consistent within each mAb type and were also consistent with the competition ELISA experiments¹³, with peptide-based epitope mapping of FVIII-C^{26,27}, as well as with ELISA assays evaluating binding of the mAbs to the following FVIII muteins: F2196L, K2227E, M2199I/F2200L, V2223A/K2227E and M2199I/F2200L/L2251V/L2252F¹³. A recent analysis of antibodies purified from a subject with an autoimmune response to FVIII (acquired hemophilia A), indicated that these included antibodies with epitopes similar to those recognized by ESH4 (Type A) and ESH8 (Type C)²⁸. The bleeding phenotype of acquired hemophilia A is often more severe than that of congenital severe hemophilia A, possibly because these antibodies bind to a somewhat different set of immunodominant B-cell epitopes and block FVIII functionality more effectively.

The strategy chosen to identify specific residues as members of a B-cell epitope by SPR was to compare the experimental dissociation rate constant k_{d} for a given FVIII-C2 mutein with the k_{d} for WT-FVIII-C2 dissociating from the same antibody, noting which substitutions increased the k_{d} to >2.0× that of WT-FVIII-C2. Once one or more muteins with this property were identified, the FVIII-C2 structure was visualized using PyMOL²³. Care was taken to analyze the FVIII-C2 muteins with substitutions in close proximity to the initial cluster of surface-exposed residues identified by their altered k_{d} values, but it was not considered essential to analyze the entire series of muteins for each antibody. The precise mapping of this series of B-cell epitopes, combined with the earlier analysis of the biochemical events (intrinsic tenase assembly, proteolytic activation of FVIII, etc.) that were blocked by each type of inhibitor¹³, pinpointed specific surfaces on the FVIII protein that interact with its partners in promoting blood coagulation. The lists of residues comprising these epitopes (Table 1) are not comprehensive, because not all surface residues were mutated, and several substitutions affected protein stability so were not analyzed further. However, the coverage of the protein surface was sufficient to definitively identify distinct clusters of residues contributing to specific antigen-antibody binding avidities.

The type A, AB and B inhibitors interfere with FVIII or FVIIIa binding to phosphatidylserine-containing phospholipid membranes¹³. As expected, the epitopes recognized by the AB and B mAbs included the hydrophobic beta hairpin turns as well as residues H2315-Q2316, which are known to participate in membrane binding^{25,29}. ELISA experiments reported earlier¹³ showed that the epitope recognized by Type AB inhibitor I109 includes residues M2199 and F2200, and the SPR results confirm that this mAb also binds to the second hairpin turn containing L2251 and L2252. Interestingly, the epitopes recognized by the Type A inhibitors ESH4, I54 and 3E6 are poised just above these projecting hairpin turns, and their inclusion of charged residues (E2181, D2187, K2206, K2207) indicates that these inhibitors block electrostatic interactions that could otherwise form between the positively charged FVIII side chains and negatively charged membrane surfaces. The kinetics of FVIII neutralization by inhibitory antibodies have long been classified as Type I or Type II^{30,31}. Type I inhibitors completely block FVIII activity at saturating concentrations, whereas Type II inhibitors do not completely inhibit clotting, even at saturating levels. Mabs I54 and 3E6 are Type I inhibitors, while ESH4 is a type II inhibitor. The three Type A epitopes identified by SPR are highly similar (FIG. 2). The different inhibition kinetics might be a result of binding to FVIII with different affinities/avidities or with a slightly different orientation that does not completely preclude FVIIIa assembly into the intrinsic tenase complex. The patient-derived inhibitory mAb BO2C11 is a Type AB antibody that buries a large surface area on the FVIII C2 domain^{14,24}. A crystal structure of a BO2C11-Fab fragment to FVIII-C2 identified 15 FVIII side chains that contacted the antibody²⁴. However, SPR-based analysis of BO2C11 binding to a series of FVIII-C2 muteins has shown that fewer than half of these side chains contributed sufficient binding avidity to be considered part of this “functional” B-cell epitope³². Similarly, we expect that the B-cell epitope residues identified herein comprise a subset of the actual contact areas between each inhibitory antibody and FVIII. This expectation has been confirmed, in part, by recent crystallographic studies illustrating the epitope recognized by another Type BC inhibitor (mAb G99)^{33,34}, and by an NMR/mass spectrometry study that identified several FVIII-C2 surfaces occluded by binding of four neutralizing mAbs³⁵. SPR-based assignments of epitope residues presented herein are consistent with the regions identified using the same or similar mAbs and these other techniques. The advantage of the mutagenesis-plus-SPR methodology is that it accurately pinpoints specific amino acid side chains that contribute significant binding avidity, distinguishing them from “bystander” residues at the protein-protein interface and thus identifying promising targets for B-cell epitope modification.

Type C and BC inhibitors including ESH8, 2-117, 2-77 and 3G6 do not prevent FVIII binding to phospholipids or to von Willebrand factor (vWF). ESH8 slows the release of thrombin-activated FVIII from vWF³⁶, and a similar mode of inhibition has been reported for IgG from an inhibitor subject³⁷, demonstrating the physiological relevance of this inhibitory mechanism. ESH8 is inhibitory only in the presence of vWF. The epitope for ESH8 has been localized by immunoblotting to FVIII residues 2248-2285³⁸. ESH8 blocks FVIII activation by FXa³⁹ and patient-derived antibodies with a similar immunoblot profile have been shown to block FVIII activation by thrombin⁴⁰. Low molecular weight peptide decoys that mimic ESH8 epitopes have been used to map this antibody's epitope, identifying the FVIII regions 2231-2240 and 2267-2270^{27,41}. Recently, peptide array analysis localized this epitope to residues 2265-2280⁴¹. The SPR results reported herein confirm that residues T2272, L2273, V2280 and V2282 comprise part of this epitope. The ESH8 epitope also includes residues H2309 and Q2311, which are distal in the linear amino acid sequence of FVIII but adjacent to the other epitope residues on the protein surface. This surface is on the opposite side of the C2 domain from the Type A epitopes (FIGS. 2 and 3). The epitope recognized by the other type C mAb, 2-117, maps to a similar surface on FVIII-C2. Unlike ESH8, however, it includes residues H2269 and Q2270, which are on a loop adjacent to the ESH8 epitope, and R2307. Unlike ESH8, 2-117 is only weakly inhibitory¹³. Possibly its binding to this loop precludes stabilization of the FVIII-vWF complex and/or the facilitated removal of thrombin-cleaved FVIIIa that has been hypothesized for the other Type C inhibitor, ESH8³⁶. The substitution R2220A affected binding to type C mAbs 2-117 and ESH8, increasing their k_{d}constants by 3.0× and 4.5×, respectively. R2220 is not conformationally contiguous with the other residues identified by SPR, therefore it was identified as an “outlier” and not included as part of these functional epitopes. The epitopes recognized by Type BC mAbs 2-77 and 3G6 overlap the Type C epitopes but also include N2225 and E2228, which are part of Type B epitopes and are further down the “back” face of FVIII-C2 towards the membrane-binding surface. Type C and BC antibodies inhibit FVIII activation by thrombin and/or FXa, but most of those analyzed to date did not compete effectively for FVIII binding to vWF¹³. These antibodies are considered “non-classical” inhibitors because their identification pointed to a previously under-appreciated and important role for the FVIII-C2 domain in proteolytic activation of FVIII. Their localization at a surface distinct from the other types of epitopes, and also on an outer surface of the FVIII protein (i.e. not at an interdomain interface) is shown in FIGS. 3A and 3B.

The mapping of this series of FVIII-C2 domain epitopes will facilitate additional studies to model the domain orientations in FVIII (which may well differ among the solution, vWF-bound and membrane-bound FVIII structures)^{21,22,42}, as well as the interactions between FVIIIa and the other components of the intrinsic tenase complex. The high-resolution definition of these physiologically relevant and medically important epitopes also suggests specific sites at which the FVIII sequence could be modified to generate less antigenic FVIII variants. We propose that next-generation therapeutic FVIII proteins could include rationally designed variants of human FVIII that, similar to porcine FVIII, could provide at least short-term hemostatic support in patients with high-titer inhibitors^{5,6}. The C2 domain of porcine FVIII contains 32 residues that differ from the human FVIII sequence⁴³. Soluble FVIII-C2 proteins with alanine substitutions at 20 of these sites were characterized by SPR. The human residues at 14 of these sites were identified as contributing to B-cell epitopes recognized by neutralizing anti-FVIII antibodies (FIG. 3C).

Both antigen-antibody affinities^{44,45} and residence times^{16,19} (which reflect avidity of binding) of these complexes are of interest in characterizing inhibitor responses. Amino acid substitutions at the antigen-antibody interfaces decreased some of the binding affinities (ΔΔG values) by 0-18 kJ/mol (Table 2). Because k_{d}s are directly related to residence times and dissociation half-lives (half-life=ln 2/k_{d}), and hence the expected duration of FVIII neutralization by inhibitor antibodies, functional B-cell epitopes were identified on the basis of the effects of amino acid substitutions on k_{d} constants. The results presented herein may be used to target functional B-cell epitopes, including critical residues in antigenic loops in the FVIII A2 domain and in other regions of FVIII⁴⁶⁻⁵², in designing novel FVIII muteins that could provide useful bypass therapy options for inhibitor patients. Because their sequences would be closer to that of the FVIII used to treat the original bleeding disorder, the risk of provoking new T-cell responses and subsequent new inhibitors⁵³⁻⁵⁸ to such rationally designed therapeutic FVIII muteins might also be lowered, in comparison with porcine FVIII used as bypass therapy. We expect that sequence modifications to neutralize immunodominant B-cell and T-cell epitopes will eventually be a feature of therapeutic FVIII proteins targeted to patients with refractory inhibitor responses, as well as to patients with poor prognostic factors such as high-risk F8 gene mutations or a family history of inhibitors⁵⁹.

Table 1.

Criteria for assigning residues to B-cell epitopes: (1) FD=East Dissociation. The k_{d}(mutein) was >2.0× the k_{d} for WT-FVIII-C2 binding to this mAb. (2) QFD=Qualitative Fast Dissociation. In these cases, the k_{d} could be estimated (by visual inspection) as >2.0× the k_{d} for WT-FVIII-C2, but the quality of the sensorgram was insufficient to fit the data to theoretical binding curves as required to determine accurate kinetic constants for these interactions. (3) NB=Non-Binding. These amino acid substitutions completely abrogated binding to the antibody. Shaded cells: These alanine substitutions resulted in k_{d}(mutein)>2.0× the k_{d} for WT-FVIII-C2 binding to this mAb, but these residues were not conformationally contiguous with the other surface-exposed candidate residues identified using the same kinetic criterion. Therefore, these “outlier” residues were not assigned to the corresponding epitopes. These substitutions likely had a localized effect on the stability/conformation of the protein, rather than disrupting specific interactions between the native FVIII side chains and the mAb. Twenty-two of the 60 FVIII-C2 muteins tested did not show altered binding kinetics to any of the mAbs (Table 2) and therefore were not assigned to any of the functional B-cell epitopes.

TABLE 1

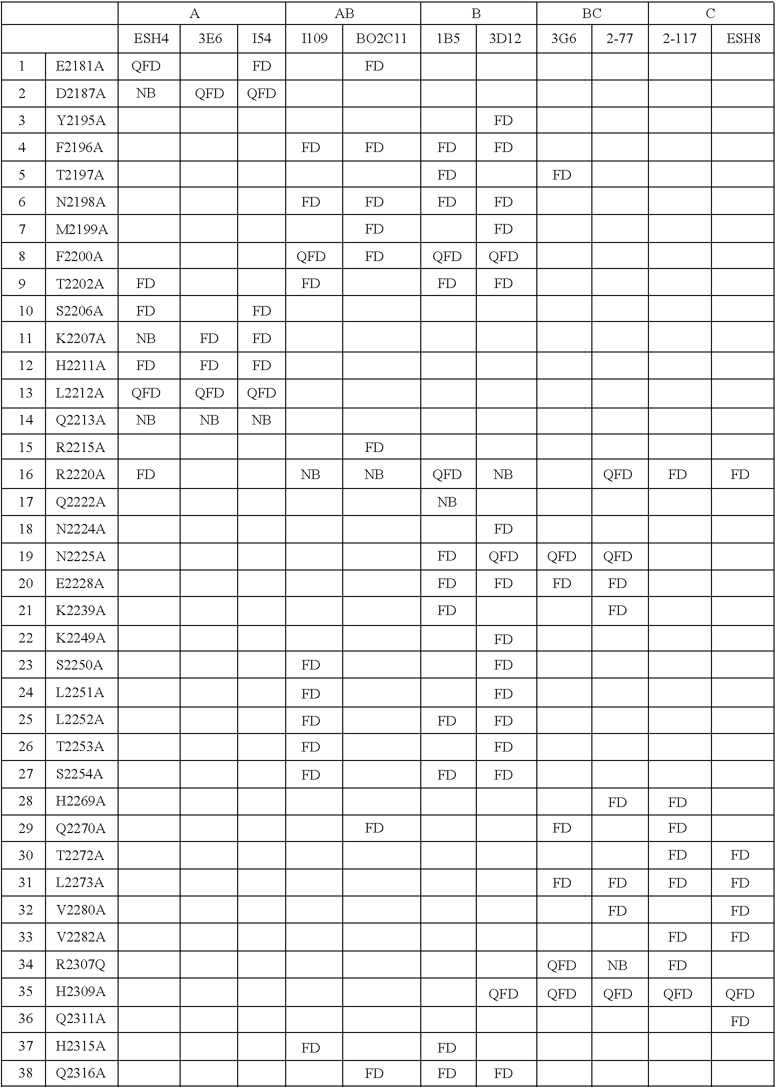

Table 2.

Each FVIII-C2 protein was tested for binding to 3 mAbs (immobilized in 3 channels of the Biacore biosensor) per SPR experiment. The goat anti-mouse IgG, Fc-γ fragment was immobilized covalently on all flow cells for capture, with the 1st flow cell serving as a reference. kₐ=association rate constant. k_{d}=dissociation rate constant. WT=WT-FVIII-C2. Mut=FVIII-C2 mutein. The kₐ and k_{d} rate constants for WT-FVIII-C2 binding to each mAb were measured during each series of experiments and the resulting k_{d}(WT) values used to calculate the k_{d}(Mut)/k_{d}(WT) ratios. The average values and standard deviations for WT-FVIII-C2 kinetic constants determined over several SPR runs are listed for each mAb. For all mAbs except BO2C11 the individual k_{d}(WT) constants, not the averaged k_{d}(WT) constants, were used to calculate the thermodynamic values for the muteins. The association of mAb BO2C11 with most of the FVIII-C2 proteins was too fast to calculate accurate kₐ rate constants under these conditions, and therefore the thermodynamic values calculated for this mAb should be interpreted with caution; the k_{d} values, however, could be measured accurately. K_{D}=k_{d}/kₐ. ΔG=−RT ln K_{D}. ΔΔG=ΔG(mutein)−ΔG(WT). k_{d}(Mut)/k_{d}(WT)=ratio of k_{d} values for the FVIII-C2 mutein versus WT-FVIII-C2. Approximate k_{d}(Mut)/k_{d}(WT) ratios (e.g. <1.0 or >2.0) were estimated for several cases where either the WT-FVIII-C2 or the FVIII-C2 mutein had an unusually fast kₐ and/or slow k_{d} constant. Residence time=1/k_{d}. Dissociation half-life=ln 2/k_{d}. (Physiological half-lives in the circulation could differ, however, e.g. due to active clearance mechanisms of FVIII and/or anti-FVIII antibodies). NB: No Binding to the biosensor surface. If this mutein bound with the same affinity as WT-FVIII-C2 to one or more mAbs that recognized a different epitope, this indicated the protein was folded properly, and the wild-type residue at this site was assigned to the B-cell epitope recognized by the antibody with altered binding kinetics. NA: Not Analyzed: the ΔΔG could not be determined with confidence because the ΔG values of WT-FVIII-C2 versus FVIII-mutein binding to the mAb were too close, or because the SPR data were qualitative or outside the limits that could be measured accurately. QFD: Qualitative Fast Dissociation; the kinetic data for this mutein could not be fitted adequately to a theoretical 1:1 binding curve, nevertheless qualitative inspection of the sensorgram clearly indicated that it had a faster k_{d} than did WT-FVIII-C2 for binding to this antibody. The residence time and hence half-life for this antigen-antibody complex is indicated as “shorter” than that of WT-FVIII-C2. The kinetic range for the Biacore T100 is: kₐ constants from ˜10³-10⁷ M⁻¹ s⁻¹; k_{d} constants from ˜10⁻⁵-0.5 s⁻¹. For fast associations with fitted kₐ≧3.2E+06, the evaluation software warns that the value approaches the limit that can be measured by the instrument. All kₐ values between 1.0E+07 and 3.0E+07 and kinetic/thermodynamic data calculated using these kₐ values are underlined. All fitted kₐ values>3.0E+07 are indicated as “>3.0E+07” and the corresponding K_{D}, ΔG and ΔΔG values were not calculated. For slow dissociations with fitted k_{d}≦3.0E-05, the evaluation software warns that the value approaches the limit that can be measured by the instrument. All k_{d} values between 1.0E-05 and 3.0E-05 and kinetic/thermodynamic data calculated using these k_{d} values are underlined. All fitted k_{d} values <1.0E-05 are indicated as “<1.0E-05”, the corresponding K_{D}, ΔG and ΔΔG values were not calculated, and the corresponding k_{d}(Mut)/k_{d}(WT) ratios are listed as ≦1.0. The residence times for these antigen-antibody complexes are indicated as “longer” than that of WT-FVIII-C2. Shaded cells indicate amino acids identified as candidates for B-cell epitopes on the basis of altered binding kinetics, as described in the FIG. 2 legend. Shaded cells correspond to the residues indicated in FIG. 2. Some residues were identified as “outliers” after visual inspection of their location on the molecular surface revealed that they were not conformationally contiguous with the other residues comprising the epitope recognized by a particular mAb. Non-contiguous outlier candidate residues are indicated by an asterisk in column 2. These residues are not included in the epitopes pictured in FIGS. 2 and 3.

TABLE 3

| Mutation | Forward Primer | Reverse Primer |
|---|---|---|
| S2175A | GGATTTAAATAGTTGCGCCATGCCATTGGGAATGGAGA | GCTTTACTCTCCATTCCCAATGGCATGGCGCAACTATTTAA |
| | GTAAAGC | ATCC |
| M2176A | GGATTTAAATAGTTGCAGCGCGCCATTGGGAATGGAGA | GCTTTACTCTCCATTCCCAATGGCGCGCTGCAACTATTTAA |
| | GTAAAGC | ATCC |
| E2181A | GCATGCCATTGGGAATGGCGAGTAAAGCAATATCAGAT | GCATCTGATATTGCTTTACTCGCCATTCCCAATGGCATGC |
| | GC | |
| S2182A | GCAGCATGCCATTGGGAATGGAGGCTAAAGCAATATCA | GCATCTGATATTGCTTTAGCCTCCATTCCCAATGGCATGCT |
| | GATGC | GC |
| S2186A | GGGAATGGAGAGTAAAGCAATAGCAGATGCACAGATTA | GCAGTAATCTGTGCATCTGCTATTGCTTTACTCTCCATTCC |
| | CTGC | C |
| D2187A | GGGAATGGAGAGTAAAGCAATATCAGCTGCACAGATTA | GCAGTAATCTGTGCAGCTGATATTGCTTTACTCTCCATTCC |
| | CTGC | C |
| T2191A | GCAATATCAGATGCACAGATTGCTGCTTCATCCTACTTT | GGTAAAGTAGGATGAAGCAGCAATCTGTGCATCTGATATT |
| | ACC | GC |
| Y2195A | GCACAGATTACTGCTTCATCCGCCTTTACCAATATGTTT | GGTGGCAAACATATTGGTAAAGGCGGATGAAGCAGTAATC |
| | GCCACC | TGTGC |
| F2196A | GCACAGATTACTGCTTCATCCTACGCTACCAATATGTTT | GGTGGCAAACATATTGGTAGCGTAGGATGAAGCAGTAATC |
| | GCCACC | TGTGC |
| T2197A | GCTTCATCCTACTTTGCCAATATGTTTGCCACCTGG | CCAGGTGGCAAACATATTGGCAAAGTAGGATGAAGC |
| N2198A | CAGATTACTGCTTCATCCTACTTTACCGCTATGTTTGCC | CCAGGTGGCAAACATAGCGGTAAAGTAGGATGAAGCAGT |
| | ACCTGG | AATCTG |
| M2199A | CTTCATCCTACTTTACCAATGCGTTTGCCACCTGGTCTC | AAGGAGACCAGGTGGCAAACGCATTGGTAAAGTAGGATG |
| | CTT | AAG |
| F2200A | CTTCATCCTACTTTACCAATATGGCTGCCACCTGGTCTC | GGAGACCAGGTGGCAGCCATATTGGTAAAGTAGGATGAA |
| | C | G |
| T2202A | CCTACTTTACCAATATGTTTGCCGCCTGGTCTCCTTCAA | GCTTTTGAAGGAGACCAGGCGGCAAACATATTGGTAAAGT |
| | AAGC | AGG |
| S2206A | GCCACCTGGTCTCCTGCAAAAGCTCGACTTCACCTCCA | CCCTTGGAGGTGAAGTCGAGCTTTTGCAGGAGACCAGGT |
| | AGGG | GGC |
| K2207A | GGTCTCCTTCAGCAGCTCGACTTCACCTCCAAGGG | CCCTTGGAGGTGAAGTCGAGCTGCTGAAGGAGACC |
| H2211A | CCTTCAAAAGCTCGACTTGCCCTCCAAGGGAGGAGTAA | GGCATTACTCCTCCCTTGGAGGGCAAGTCGAGCTTTTGAA |
| | TGCC | GG |
| L2212A | CCTTCAAAAGCTCGACTTCACGCCCAAGGGAGGAGTA | GGCATTACTCCTCCCTTGGGCGTGAAGTCGAGCTTTTGAA |
| | ATGCC | GG |
| Q2213A | CCTTCAAAAGCTCGACTTCACCTCGCAGGGAGGAGTAA | GGCATTACTCCTCCCTGCGAGGTGAAGTCGAGCTTTTGAA |
| | TGCC | GG |
| R2215A | CGACTTCACCTCCAAGGGGCGAGTAATGCCTGGAGAC | GGTCTCCAGGCATTACTCGCCCCTTGGAGGTGAAGTCG |
| | C | |
| R2220A | CCAAGGGAGGAGTAATGCCTGGGCACCTCAGGT | ACCTGAGGTGCCCAGGCATTACTCCTCCCTTGG |
| Q2222A | GGAGTAATGCCTGGAGACCTGCGGTGAATAATCCAAAA | CCACTCTTTTGGATTATTCACCGCAGGTCTCCAGGCATTA |
| | GAGTGG | CTCC |
| V2223A | GCCTGGAGACCTCAGGCGAATAATCCAAAAGAGTGG | CCACTCTTTTGGATTATTCGCCTGAGGTCTCCAGGC |
| N2224A | GGAGTAATGCCTGGAGACCTCAGGTGGCTAATCCAAA | GCCACTCTTTTGGATTAGCCACCTGAGGTCTCCAGGCATT |
| | AGAGTGGC | ACTCC |
| N2225A | CCTGGAGACCTCAGGTGAATGCTCCAAAAGAGTGGCT | GCAGCCACTCTTTTGGAGCATTCACCTGAGGTCTCCAGG |
| | GC | |
| E2228A | CAGGTGAATAATCCAAAAGCGTGGCTGCAAGTGGACTT | GAAGTCCACTTGCAGCCACGCTTTTGGATTATTCACCTG |
| | C | |
| Q2235A | GGCTGCAAGTGGACTTCGCGAAGACAATGAAAGTCAC | CCTGTGACTTTCATTGTCTTCGCGAAGTCCACTTGCAGCC |
| | AGG | |
| K2239A | GGACTTCCAGAAGACAATGGCAGTCACAGGAGTAACTA | GAGTAGTTACTCCTGTGACTGCCATTGTCTTCTGGAAGTC |
| | CTC | C |
| K2249A | GTAACTACTCAGGGAGTAGCATCTCTGCTTACCAGCAT | CACATACATGCTGGTAAGCAGAGATGCTACTCCCTGAGTA |
| | GTATGTG | GTTAC |
| S2250A | CAGGGAGTAAAAGCTCTGCTTACCAGCATGTATGTG | CACATACATGCTGGTAAGCAGAGCTTTTACTCCCTG |
| L2251A | CTACTCAGGGAGTAAAATCTGCGCTTACCAGCATGTAT | CCTTCACATACATGCTGGTAAGCGCAGATTTTACTCCCTG |
| | GTGAAGG | AGTAG |
| L2252A | CAGGAGTAAAATCTCTGGCTACCAGCATGTATGTGAAG | CTCCTTCACATACATGCTGGTAGCCAGAGATTTTACTCCCT |
| | GAG | G |
| T2253A | GGAGTAAAATCTCTGCTTGCCAGCATGTATGTGAAGGA | CTCCTTCACATACATGCTGGCAAGCAGAGATTTTACTCC |
| | G | |
| S2254A | GGAGTAAAATCTCTGCTTACCGCCATGTATGTGAAGGA | GAGGAACTCCTTCACATACATGGCGGTAAGCAGAGATTTT |
| | GTTCCTC | ACTCC |
| E2259A | GCATGTATGTGAAGGCGTTCCTCATCTCCAGC | GCTGGAGATGAGGAACGCCTTCACATACATGC |
| Q266A | CCTCATCTCCAGCAGTGCAGATGGCCATCAGTGGAC | GTCCACTGATGGCCATCTGCACTGCTGGAGATGAGG |
| H2269A | CATCTCCAGCAGTCAAGATGGCGCTCAGTGGACTCTC | GAGAGTCCACTGAGCGCCATCTTGACTGCTGGAGATG |
| Q2270A | GCAGTCAAGATGGCCATGCGTGGACTCTCTTTTTTCAG | GGCATTCTGAAAAAAGAGAGTCCACGCATGGCCATCTTGA |
| | AATGCC | CTGC |
| T2272A | GGCCATCAGTGGGCTCTCTTTTTTCAGAATGGC | GCCATTCTGAAAAAAGAGAGCCCACTGATGGCC |
| L2273A | GATGGCCATCAGTGGACTGCCTTTTTTCAGAATGGCAA | CTTTACTTTGCCATTCTGAAAAAAGGCAGTCCACTGATGG |
| | AGTAAAG | CCATC |
| Q2276A | GGCCATCAGTGGACTCTCTTTTTTGCGAATGGCAAAGT | CCTTTACTTTGCCATTCGCAAAAAAGAGAGTCCACTGATG |
| | AAAGG | GCC |
| N2277A | CAGTGGACTCTCTTTTTTCAGGCTGGCAAAGTAAAGGT | CTGAAAAACCTTTACTTTGCCAGCCTGAAAAAAGAGAGTC |
| | TTTTCAG | CACTG |
| V2280A | GGACTCTCTTTTTTCAGAATGGCAAAGCAAAGGTTTTTC | CCATTCTGAAAAACCTTTGCTTTGCCATTCTGAAAAAAGAG |
| | AGAATGG | AGTCC |
| V2282A | CAGAATGGCAAAGTAAAGGCTTTTCAGAATGGCAAAGT | CCTTTACTTTGCCATTCTGAAAAGCCTTTACTTAGCCATTC |
| | AAAGG | TG |
| Q2284A | CAGAATGGCAAAGTAAAGTTTTTGCGAATGGCAAAGT | CCTTTACTTTGCCATTCGCAAAAACCTTTACTTTCCATTC |
| | AAAGG | TG |
| Q2287A | GTAAAGGTTTTTCAGGGAAATGCAGACTCCTTCACACC | CACCACAGGTGTGAAGGAGTCTGCATTTCCCTGAAAAACC |
| | TGTGGTG | TTTAC |
| D2288A | GGTTTTTCAGGGAAATCAAGCCTCCTTCACACCTGTGG | CCACAGGTGTGAAGGAGGCTTCATTTCCCTGAAAAACC |
| F2290A | GGAAATCAAGACTCCGCCACACCTGTGGTGAACTCTCT | |
| | AG | CTAGAGAGTTCACCACAGGTGTGGCGGAGTCTTGATTTCC |
| T2291A | GGAAATCAAGACTCCTTCGCACCTGTGGTGAACTC | GAGTTCACCACAGGTGCGAAGGAGTCTTGATTTCC |
| V2294A | CCTTCACACCTGTGGCGAACTCTCTAGACCCC | GGGGTCTAGAGAGTTCGCCACAGGTGTGAAGG |
| 322961 | CCTGTGGTGAACGCTCTAGACCCACCGTTACTG | CAGTAACGGTGGGTCTAGAGCGTTCACCACAGG |
| P22991 | CCTGTGGTGAACTCTCTAGACGCACCGTTACTGACTCG | CGAGTCAGTAACGGTGCGTCTAGAGAGTTCACCACAGG |
| L23021 | GACCCACCGTTAGCGACTCGCTACCTTCGAATTCACC | CAGTAGAGGTCCGCTGCCTCGCAGCCCAGAACC |
| R23070 | CTGACTCGCTACCTTCAAATTCACCCCCAGAGTTGG | CCAACTCTGGGGGTGAATTTGAAGGTAGCGAGTCAG |
| H2309A | CGCTACCTTCGAATTGCCCCCCAGAGTTGGGTGC | GCACCCAACTCTGGGGGGCAATTCGAAGGTAGCG |
| Q2311A | CCTTCGAATTCACCCCGCGAGTTGGGTGCACCAG | CTGGTGCACCCAACTCGCGGGGTGAATTCGAAGG |
| H2315A | CCAGAGTTGGGTGGCCCAGATTGCCCTGAGGATGG | CCATCCTCAGGGCAATCTGGGCCACCCAACTCTGG |
| Q23161 | CCCCAGAGTTGGGTGCACGCCATTGCCCTGAGGATGG | CCATCCTCAGGGCAATGGCGTGCACCCAACTCTGGGG |
| E23271 | GGTTCTGGGCTGCGCGGCACAGGACC | GGTCCTGTGCCGCGCAGCCCAGAACC |
| Q2329A | GGTTCTGGGCTGCGAGGCAGCGGACCTCTACTG | CAGTAGAGGTCCGCTGCCTCGCAGCCCAGAACC |

### Example 2

### Administration of a Modified Factor VIII to a Mammal in Need Thereof

Mammals (e.g., mice, rats, rodents, humans, guinea pigs) are used in the study. Mammals are administered (e.g., intravenously) one or more modified factor VIIIs described herein or a control. In some instances the modified factor VIII is a modified factor VIII polypeptide described in the summary section above. The modified factor VIII can be any of those disclosed herein. Various types of modifications can be used, e.g., additions, delections, substitutions, and/or chemical modifications. In some instances the modified factor VIII is formulated in a pharmaceutically acceptable carrier. In some instances the modified factor VIII is formulated as described in the pharmaceutical compositions section above, e.g., using the same methods and dosages used for administration of an unmodified factor VIII.

Multiple rounds of doses are used where deemed useful. Effects on factor VIII-specific immune responses, inflammatory cytokine levels, and/or conditions associated with hemophilia are monitored in the mammals, e.g., via tetramer analysis, ELISA, and other methods known in the art. Similar studies are performed with different treatment protocols and administration routes (e.g., intramuscular administration, etc.). The effectiveness of a modified factor VIII is demonstrated by measuring the anti-FVIII antibody titer (either absolute titer or neutralizing activity titer, the latter measured in Bethesda units/mL). Effectiveness may also be measured by measuring FVIII half-life, relative affinity FVIII binding to von Willebrand factor, phospholipids or platelets, and binding to other serine proteases in the coagulation cascade, or by comparing the factor VIII-specific immune responses, inflammatory cytokine levels, and/or conditions associated with hemophilia in mammals treated with a modified factor VIII disclosed herein to mammals treated with control formulations and/or an unmodified factor VIII.

In an example, a human subject in need of treatment is selected or identified. The subject can be in need of, e.g., reducing, preventing, or treating a condition associated with an immune response to factor VIII and/or a condition associated with hemophilia. In some cases, the subject may be a non-hemophilia A patient with an autoimmune response to their endogenous factor VIII. In some instances, the subject is a hemophilia A patient with a recall response to factor VIII; for example, an individual who had an inhibitor earlier and then developed one again later—for example, during surgery and intensive factor VIII treatment later in life. The identification of the subject can occur in a clinical setting, or elsewhere, e.g., in the subject's home through the subject's own use of a self-testing kit.

At time zero, a suitable first dose of a modified factor VIII is administered to the subject. The modified factor VIII is formulated as described herein. After a period of time following the first dose, e.g., 7 days, 14 days, and 21 days, the subject's condition is evaluated. e.g., by measuring the anti-FVIII antibody titer (either absolute titer or neutralizing activity titer, the latter measured in Bethesda units/mL). Effectiveness may also be measured by measuring FVIII half-life, relative affinity FVIII binding to von Willebrand factor, phospholipids or platelets, and binding to other serine proteases in the coagulation cascade, or by comparing the factor VIII-specific immune responses, inflammatory cytokine levels, and/or conditions associated with hemophilia in mammals treated with a modified factor VIII. Other relevant criteria can also be measured, e.g., ELISPOT. The number and strength of doses are adjusted according to the subject's needs.

After treatment, the subject's anti-FVIII antibody titer (either absolute titer or neutralizing activity titer, the latter measured in Bethesda units/mL), FVIII half-life, relative affinity FVIII binding to von Willebrand factor, levels of phospholipids or platelets, binding to other serine proteases in the coagulation cascade, factor VIII-specific immune responses, inflammatory cytokine levels, and/or conditions associated with hemophilia in mammals treated with a modified factor VIII are lowered and/or improved relative to the levels existing prior to the treatment, or relative to the levels measured in a similarly afflicted but untreated/control subject, or relative to the levels measured in a similarly afflicted subject treated with an unmodified factor Viii.

### REFERENCES

1. Ehrenforth S, Kreuz W, Scharrer I, et al. Incidence of development of factor VIII and factor IX inhibitors in haemophiliacs. Lancet. 1992; 339(8793):594-598.
2. Kreuz W, Ettingshausen C E, Zyschka A, et al. Inhibitor development in previously untreated patients with hemophilia A: a prospective long-term follow-up comparing plasma-derived and recombinant products. Semin Thromb Hemost. 2002; 28(3):285-290.
3. Gouw S C, Fijnvandraat K. Unraveling the genetics of inhibitors in hemophilia. Blood. 2013; 121(8):1250-1251.
4. Cugno M, Gualtierotti R, Tedeschi A, Meroni P L. Autoantibodies to coagulation factors: From pathophysiology to diagnosis and therapy. Autoimmun Rev. 2013.
5. Aledort L M. The role of porcine factor VIII in the management of unexpected bleeding episodes. Haemophilia. 2002; 8 Suppl 1:17-19; discussion 28-32.
6. Toschi V. OBI-1, porcine recombinant Factor VIII for the potential treatment of patients with congenital hemophilia A and alloantibodies against human Factor VIII. Curr Opin Mol Ther. 2010:12(5):617-625.
7. Kulkarni R. Aledort L M, Berntorp E, et al. Therapeutic choices for patients with hemophilia and high-titer inhibitors. Am J Hematol. 2001; 67(4):240-246.
8. Kempton C L, Abshire T C, Deveras R A, et al. Pharmacokinetics and safety of OBI-1, a recombinant B domain-deleted porcine factor VIII, in subjects with haemophilia A. Haemophilia. 2012:18(5):798-804.
9. Barrow R T. Lollar P. Neutralization of antifactor VIII inhibitors by recombinant porcine factor VIII. J Thromb Haemost. 2006; 4(10):2223-2229.
10. Arai M. Scandella D, Hoyer L W. Molecular basis of factor VIII inhibition by human antibodies. Antibodies that bind to the factor VIII light chain prevent the interaction of factor VIII with phospholipid. J Clin Invest. 1989; 83(6):1978-1984.
11. Scandella D H. Nakai H, Felch M, et al. In hemophilia A and autoantibody inhibitor patients: the factor VIII A2 domain and light chain are most immunogenic. Thromb Res. 2001; 101(5):377-385.
12. Pratt K P. Relating structure to function: the role of the C2 domain in Factor VIII. Curr Opin Drug Discov Devel. 2000; 3(5):516-526.
13. Meeks S L, Healey J F, Parker E T, Barrow R T. Lollar P. Antihuman factor VIII C2 domain antibodies in hemophilia A mice recognize a functionally complex continuous spectrum of epitopes dominated by inhibitors of factor VIII activation. Blood. 2007; 110(13):4234-4242.
14. Jacquemin M G, Desqueper B G, Benhida A. et al. Mechanism and kinetics of factor VIII inactivation: study with an IgG4 monoclonal antibody derived from a hemophilia A patient with inhibitor. Blood. 1998; 92(2):496-506.
15. Karlsson R Katsamba P S, Nordin H, Pol E, Myszka D G. Analyzing a kinetic titration series using affinity biosensors. Anal Biochem. 2006; 349(1):136-147.
16. Copeland R A, Pompliano D L, Meek T D. Drug-target residence time and its implications for lead optimization. Nat Rev Drug Discov. 2006; 5(9):730-739.
17. Wood E R, Truesdale A T, McDonald O B, et al. A unique structure for epidermal growth factor receptor bound to GW572016 (Lapatinib): relationships among protein conformation, inhibitor off-rate, and receptor activity in tumor cells. Cancer Res. 2004; 64(18):6652-6659.
18. Mondal K, Regnstrom K, Morishige W, et al. Thermodynamic and kinetic characterization of hydroxyethylamine beta-secretase-1 inhibitors. Biochem Biophys Res Commun. 2013; 441(2):291-296.
19. Tummino P J, Copeland R A. Residence time of receptor-ligand complexes and its effect on biological function. Biochemistry. 2008; 47(20):5481-5492.
20. Pratt K P, Shen B W, Takeshima K, Davie E W, Fujikawa K, Stoddard B L. Structure of the C2 domain of human factor VIII at 1.5 A resolution. Nature. 1999; 402(6760): 439-442.
21. Ngo J C, Huang M, Roth D A, Furie B C, Furie B. Crystal structure of human factor VIII: implications for the formation of the factor IXa-factor VIIIa complex. Structure. 2008; 16(4):597-606.
22. Shen B W. Spiegel P C, Chang C H, et al. The tertiary structure and domain organization of coagulation factor VII. Blood. 2008; 111(3):1240-1247.
23. PyMOL. The PyMOL Molecular Graphics System, Version 1.5.0.4 Schrödinger, LLC.; 2013.
24. Spiegel P C, Jr., Jacquemin M, Saint-Remy J M, Stoddard B L, Pratt K P. Structure of a factor VIII C2 domain-immunoglobulin G4kappa Fab complex: identification of an inhibitory antibody epitope on the surface of factor VIII. Blood. 2001; 98(1):13-19.
25. Lewis D A, Moore K D, Ortel T L. Binding of factor VIII inhibitors to discrete regions of the factor VIII C2 domain disrupt phospholipid binding. Blood Coagul Fibrinolysis. 2003; 14(4):361-368.
26. Lebreton A, Moreau V, Lapalud P, et al. Discontinuous epitopes on the C2 domain of coagulation Factor VIII mapped by computer-designed synthetic peptides. Br J Haematol. 2011; 155(4):487-497.
27. Villard S, Lacroix-Desmazes S, Kieber-Emmons T, et al. Peptide decoys selected by phage display block in vitro and in vivo activity of a human anti-FVIII inhibitor. Blood. 2003; 102(3):949-952.
28. Matsumoto T, Nogami K, Ogiwara K, Shima M. A putative inhibitory mechanism in the tenase complex responsible for loss of coagulation function in acquired haemophilia A patients with anti-C2 autoantibodies. Thromb Haemost. 2012; 107(2):288-301.
29. Liu Z. Lin L, Yuan C, et al. Trp2313-His2315 of factor VIII C2 domain is involved in membrane binding: structure of a complex between the C2 domain and an inhibitor of membrane binding. J Biol Chem. 2010; 285(12):8824-8829.
30. Biggs R. Austen D E, Denson K W. Rizza C R, Borrett R. The mode of action of antibodies which destroy factor VIII. I. Antibodies which have second-order concentration graphs. Br J Haematol. 1972:23(2):125-135.
31. Biggs R, Austen D E, Denson K W, Borrett R, Rizza C R. The mode of action of antibodies which destroy factor VIII. T I. Antibodies which give complex concentration graphs. Br J Haematol. 1972:23(2):137-155.
32. Lin J C, Kaushik V, Jacquemin M, Thompson A R, Pratt K P. Six amino acid residues in a 1200 A² interface mediate binding of factor VIII to an IgG4-kappa inhibitory antibody. Blood 2008; 112:3380.
33. Walter J D, Werther R A. Polozova M S. et al. Characterization and solution structure of the factor VIII C2 domain in a ternary complex with classical and non-classical inhibitor antibodies. J Biol Chem. 2013; 288(14):9905-9914.
34. Walter J D, Werther R A, Brison C M, et al. Structure of the factor VIII C2 domain in a ternary complex with 2 inhibitor antibodies reveals classical and nonclassical epitopes. Blood. 2013; 122(26):4270-4278.
35. Sevy A M, Healey J F, Deng W, Spiegel P C, Meeks S L, Li R. Epitope mapping of inhibitory antibodies targeting the C2 domain of coagulation factor VIII by hydrogen-deuterium exchange mass spectrometry. J Thromb Haemost. 2013.
36. Saenko E L, Shima M, Gilbert G E, Scandella D. Slowed release of thrombin-cleaved factor VIII from von Willebrand factor by a monoclonal and a human antibody is a novel mechanism for factor VIII inhibition. J Biol Chem. 1996; 271(44):27424-27431.
37. Jacquemin M, Benhida A, Peerlinck K, et al. A human antibody directed to the factor VIII C1 domain inhibits factor VIII cofactor activity and binding to von Willebrand factor. Blood. 2000; 95(1):156-163.
38. Scandella D, Gilbert G E, Shima M. et al. Some factor VIII inhibitor antibodies recognize a common epitope corresponding to C2 domain amino acids 2248 through 2312, which overlap a phospholipid-binding site. Blood. 1995:86(5):1811-1819.
39. Nogami K, Shima M, Hosokawa K, et al. Role of factor VIII C2 domain in factor VIII binding to factor Xa. J Biol Chem. 1999; 274(43):31000-31007.
40. Nogami K. Shima M, Hosokawa K. et al. Factor VIII C2 domain contains the thrombin-binding site responsible for thrombin-catalyzed cleavage at Arg1689. J Biol Chem. 2000; 275(33):25774-25780.
41. Albert T, Egler C, Jakuschev S, et al. The B-cell epitope of the monoclonal anti-factor VIII antibody ESH8 characterized by peptide array analysis. Thromb Haemost. 2008; 99(3):634-637.
42. Stoilova-McPhie S, Lynch G C, Ludtke S, Pettitt B M. Domain organization of membrane-bound factor VIII. Biopolymers. 2013; 99(7):448-459.
43. Healey J F. Lubin I M, Lollar P. The cDNA and derived amino acid sequence of porcine factor VIII. Blood. 1996; 88(11):4209-4214.
44. Cunningham B C, Wells J A. Comparison of a structural and a functional epitope. J Mol Biol. 1993; 234(3):554-563.
45. Sundberg E J, Mariuzza R A. Molecular recognition in antibody-antigen complexes. Adv Protein Chem. 2002; 61:119-160.
46. Barrow R T, Healey J F, Gailani D, Scandella D, Lollar P. Reduction of the antigenicity of factor VIII toward complex inhibitory antibody plasmas using multiply-substituted hybrid human/porcine factor VIII molecules. Blood. 2000; 95(2):564-568.
47. Fay P J, Scandella D. Human inhibitor antibodies specific for the factor VIII A2 domain disrupt the interaction between the subunit and factor IXa. J Biol Chem. 1999; 274(42):29826-29830.
48. Healey J F, Lubin I M, Nakai H. et al. Residues 484-508 contain a major determinant of the inhibitory epitope in the A2 domain of human factor VIII. J Biol Chem. 1995; 270(24):14505-14509.
49. Lollar P. Mapping factor VIII inhibitor epitopes using hybrid human/porcine factor VIII molecules. Haematologica. 2000:85(10 Suppl):26-28: discussion 28-30.
50. Sawamoto Y, Shima M, Tanaka I, et al. Anti-factor VIII inhibitor alloantibodies recognizing the A2 domain in the human factor VIII heavy chain poorly bind to porcine factor VIII. Int J Hematol. 1997; 65(2):151-158.
51. Lubin I M, Healey J F, Scandella D, Runge M S, Lollar P. Elimination of a major inhibitor epitope in factor VIII. J Biol Chem. 1994; 269(12):8639-8641.
52. Lubin I M, Healey J F, Barrow R T, Scandella D, Lollar P. Analysis of the human factor VIII A2 inhibitor epitope by alanine scanning mutagenesis. J Biol Chem. 1997; 272(48):30191-30195.
53. Jacquemin M, Vantomme V, Buhot C, et al. CD4+ T-cell clones specific for wild-type factor VIII: a molecular mechanism responsible for a higher incidence of inhibitor formation in mild/moderate hemophilia A. Blood. 2003; 101(4):1351-1358.
54. Ettinger R A, James E A, Kwok W W, Thompson A R. Pratt K P. HLA-D R-restricted T-cell responses to factor VIII epitopes in a mild haemophilia A family with missense substitution A2201P. Haemophilia. 2010; 16(102):44-55.
55. Ettinger R A, James E A, Kwok W W, Thompson A R. Pratt K P. Lineages of human T-cell clones, including T helper 17/T helper I cells, isolated at different stages of anti-factor VIII immune responses. Blood. 2009; 114(7):1423-1428.
56. James E A, Kwok W W, Ettinger R A, Thompson A R, Pratt K P. T-cell responses over time in a mild hemophilia A inhibitor subject: epitope identification and transient immunogenicity of the corresponding self-peptide. J Thromb Haemost. 2007; 5(12):2399-2407.
57. Pratt K P. Thompson A R. B-cell and T-cell epitopes in anti-factor VIII immune responses. Clin Rev Allergy Immunol. 2009; 37(2):80-95.
58. Scott D W, Pratt K P, Miao C H. Progress toward inducing immunologic tolerance to factor VIII. Blood. 2013; 121(22):4449-4456.
59. Gouw S C, van den Berg H M, Oldenburg J, et al. F8 gene mutation type and inhibitor development in patients with severe hemophilia A: systematic review and meta-analysis. Blood. 2012; 119(12):2922-2934.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A modified Factor VIII polypeptide comprising at least one amino acid modification in an unmodified Factor VIII polypeptide, wherein the at least one amino acid modification is at a position corresponding to positions 2173-2332 of the C2 domain of the amino acid sequence set forth in SEQ ID NO: 1, and wherein the at least one amino acid modification is at a position corresponding to position D2187, K2207, H2211, L2212, Q2213, E2181, T2202, S2206, R2220, E2181, S2206, F2196, N2198, F2200, T2202, S2250, L2251, L2252, T2253, S2254, H2315, M2199, R2215, Q2270, Q2316, F2196, N2198, F2200, T2202, N2225, E2228, L2252, S2254, Q2316, T2197, Q2222, K2239, H2315, Y2195, M2199, N2224, K2249, S2250, L2251, T2253, H2309, N2225, E2228, L2273, R2307, H2309, T2197, Q2270, R2220, K2239, H2269, V2280, R2220, T2272, L2273, V2282, H2309, H2269, Q2270, V2280, Q2311, or R2307 of the amino acid sequence set forth in SEQ ID NO: 1.

2. The modified Factor VIII polypeptide of claim 1, wherein the at least one amino acid modification is at a position corresponding to position D2187, K2207, H2211, L2212, Q2213, E2181, T2202, S2206, R2220, E2181, or S2206 of the amino acid sequence set forth in SEQ ID NO: 1.

3. The modified Factor VIII polypeptide of claim 1, wherein the at least one amino acid modification is at a position corresponding to position E2181, D2187, K2207, Q2213, S2206, and Q2213 of the amino acid sequence set forth in SEQ ID NO: 1.

4. The modified Factor VIII polypeptide of claim 1, wherein the at least one amino acid modification is at a position corresponding to position F2196, N2198, F2200, R2220, T2202, S2250, L2251, L2252, T2253, S2254 and H2315, E2181, M2199, R2215, Q2270, or Q2316 of the amino acid sequence set forth in SEQ ID NO: 1.

5. The modified Factor VIII polypeptide of claim 1, wherein the at least one amino acid modification is at a position corresponding to position F2196, N2198, M2199, F2200, R2215, R2220, S2250, L2252, and S2254 of the amino acid sequence set forth in SEQ ID NO: 1.

6. The modified Factor VIII polypeptide of claim 1, wherein the at least one amino acid modification is at a position corresponding to position F2196, N2198, F2200, T2202, R2220, N2225, E2228, L2252, S2254, Q2316, T2197, Q2222, K2239, H2315, Y2195, M2199, N2224, K2249, S2250, L2251, T2253, or H2309 of the amino acid sequence set forth in SEQ ID NO: 1.

7. The modified Factor VIII polypeptide of claim 1, wherein the at least one amino acid modification is at a position corresponding to position F2196, N2198, T2202, R2220, Q2222, N2224, N2225, E2228, K2239, L2251, L2252, T2253, S2254, H2315, and Q2316 of the amino acid sequence set forth in SEQ ID NO: 1.

8. The modified Factor VIII polypeptide of claim 1, wherein the at least one amino acid modification is at a position corresponding to position N2225, E2228, L2273, R2307, H2309, T2197, Q2270, R2220, K2239, H2269, or V2280 of the amino acid sequence set forth in SEQ ID NO: 1.

9. The modified Factor VIII polypeptide of claim 1, wherein the at least one amino acid modification is at a position corresponding to position L2273, E2228, L2273, and R2307 of the amino acid sequence set forth in SEQ ID NO: 1.

10. The modified Factor VIII polypeptide of claim 1, wherein the at least one amino acid modification is at a position corresponding to position R2220, T2272, L2273, V2282, H2309, H2269, Q2270, V2280, Q2311, or R2307 of the amino acid sequence set forth in SEQ ID NO: 1.

11. The modified Factor VIII polypeptide of claim 1, wherein the at least one amino acid modification is at a position corresponding to position Q2270, L2273, R2307, L2273, and V2280 of the amino acid sequence set forth in SEQ ID NO: 1.

12. The modified Factor VIII polypeptide of claim 1, wherein the at least one amino acid modification is an amino acid substitution at a position of the amino acid sequence set forth in SEQ ID NO: 1, selected from the group consisting of E2181A, D2187A, Y2195A, F2196A, T2197A, N2198A, M2199A, F2200A, T2202A, S2206A, K2207A, H2211A, L2212A, Q2213A, R2215A, R2220A, Q2222A, N2224A, N2225A, E2228A, K2239A, K2249A, S2250A, L2251A, L2252A, T2253A, S2254A, H2269A, Q2270A, T2272A, L2273A, V2280A, V2282A, R2307Q, H2309A, Q2311A, H2315A, and Q2316A.

13. The modified Factor VIII polypeptide of claim 1, wherein the at least one amino acid modification is F2196K or F2196A.

14. (canceled)

15. The modified Factor VIII polypeptide of claim 1, wherein the at least one amino acid modification is an amino acid deletion.

16. The modified Factor VIII polypeptide of claim 1, wherein the at least one amino acid modification is an amino acid addition.

17. The modified Factor VIII polypeptide of claim 1, wherein the at least one amino acid modification is an amino acid substitution.

18.-28. (canceled)

29. The modified Factor VIII polypeptide of claim 1, wherein the modified Factor VIII polypeptide further comprises at least one additional amino acid modification.

30. The modified Factor VIII polypeptide of claim 29, wherein the at least one additional amino acid modification is a modification in a T cell epitope.

31.-40. (canceled)

41. A method of making the modified Factor VIII polypeptide of claim 1, comprising: providing a host cell comprising a nucleic acid sequence that encodes the modified Factor VIII polypeptide; and maintaining the host cell under conditions in which the modified Factor VIII polypeptide is expressed.

42. A method for reducing or preventing a condition associated with an immune response to Factor VIII, comprising administering to a subject in need thereof an effective amount of the modified Factor VIII polypeptide of claim 1.

43.-50. (canceled)
